(19) United States
(12) Patent Application Publication
(10) Pub. No.: US 2024/0398943 A1
(43) Pub. Date: Dec. 5, 2024

(54) METHODS OF TREATING CANCER AND THE PHARMACEUTICAL COMPOSITIONS THEREOF

(71) Applicant: SYSTIMMUNE, INC., Redmond, WA (US)

(72) Inventors: Sa XIAO, Chengdu, Sichuan (CN); Muran DING, Chengdu, Sichuan (CN); Yong ZHANG, Chengdu, Sichuan (CN); Shi ZHUO, Chengdu, Sichuan (CN); Nga Sze Amanda MAK, Mukilteo, WA (US); Jahan KHALILI, Kirkland, WA (US); Hai ZHU, Chengdu, Sichuan (CN); Yi ZHU, Redmond, WA (US)

(21) Appl. No.: 18/693,507

(22) PCT Filed: Oct. 3, 2022
(86) PCT No.: PCT/US2022/077490

§ 371 Date: Mar. 19, 2024

Related U.S. Application Data
(60) Provisional application No. 63/251,664, filed on Oct. 3, 2021.

(51) Int. Cl. A61K 39/395; A61K 31/282; A61K 31/337; A61K 31/4745; A61K 31/506; A61K 31/519; A61K 33/243; A61K 39/00; A61P 35/00

(52) U.S. Cl. CPC A61K 39/3955; A61K 31/282; A61K 31/337; A61K 31/4745; A61K 31/506; A61K 31/519; A61K 33/243; A61P 35/00; A61K 2039/505; A61K 2039/545; A61K 2039/55

(57) ABSTRACT

A method for treating cancer in a subject comprising, administering to the subject a bispecific antibody having a binding specificity to EGFR and HER3 and a therapeutic agent, wherein the therapeutic agent comprises a tyrosine kinase inhibitor (TKI), an alkylating agent, an anti-metabolite, an anti-microtubule agent, a cytotoxic antibiotic, a topoisomerase inhibitor, a chemoprotectant, or a combination thereof.

### CROSS REFERENCE TO RELATED APPLICATIONS

This application claims the benefit of the filing date of U.S. Provisional Application Ser. No. 63/251,664 filed Oct. 3, 2021, under 35 U.S.C. 119(e), the entire disclosures of which are incorporated by reference herein.

### TECHNICAL FIELD

The present application relates to combination therapies useful for the treatment of cancer. In particular, the present application relates to a combination therapy which comprises a bispecific antibody, which specifically binds to human EGFR and HER3, and a chemotherapeutic agent, including a tyrosine kinase inhibitor.

### BACKGROUND

Unless otherwise indicated herein, the materials described in this section are not prior art to the claims in this application and are not admitted being prior art by inclusion in this section.

The human epidermal growth factor receptor family contains four receptor tyrosine kinases (EGFR/HER1, HER2, HER3, and HER4). EGFR is extensively studied for its roles in gene expression, cell proliferation, adhesion, angiogenesis, apoptosis, and tumor metastasis. EGFR is overexpressed in over 90% of head and neck squamous cell carcinoma (HNSCC). High levels of EGFR mutations are found in non-small cell lung cancer (NSCLC). While both EGFR and HER3 are frequently upregulated and/or mutated in a variety of tumors, HER3 is a catalytically impaired member of the EGFR family that contributes to the heterodimeric complex formed by the kinase domains of EGFR, HER3, and HER2. The cancer associated HER3 mutations enhance the allosteric activator function of HER3 by repurposing local interactions at the dimerization interface.

In cases of HNSCC patients, the standard care, such as postoperative radiation therapy with concurrent cisplatin-based chemotherapy, improves locoregional control and disease-free survival. But the overall survival rate remains at about 50%.

Several tyrosine kinase inhibitors (TKIs) are developed as targeted chemotherapy to inhibit receptor tyrosine kinase activities in human cancer cells. TKIs may be used as single agent therapy targeting the intracellular kinase domain of EGFR signaling. For example, gefitinib (IRESSA®, AstraZeneca) is approved for treating NSCLC with EGFR exon 19 deletions (exon19del) or exon 21 (L858R) substitution mutations. The clinical benefit from TKI treatment either as a single agent or in combination with radiation treatment seems to be limited to 10-15% of HNSCC patients. The combination of EGFR-TKIs targeted chemotherapy and standard chemotherapy is under investigation in HNSCC patients (Rebuzzi et al., 2019). These approaches are likely to have their own limitations. For example, a therapeutic dose in chemotherapeutic combinations may have significant toxicity due to its non-specific mechanism of action, which becomes a limiting factor to the efficacy of the treatment plan.

Single agent targeted chemotherapy (such as TKIs) or targeted immune antibody therapy that demonstrates initial efficacy does not lead to durable cancer control (Wu et al., 2020). In case of targeted immune therapy, Cetuximab (ERBITUX®), a monoclonal antibody (mAb) targeting the extracellular domain of EGFR, has been approved as a monotherapy for clinical indications of HNSCC, namely, recurrent or metastatic HNSCC progressing after platinum-based therapy, local/regional advanced HNSCC in combination with radiation therapy, late-stage HNSCC in combination with chemotherapy, and recurrent locoregional disease or metastatic HNSCC in combination with platinum-based therapy with Fluorouracil. However, the response rate for Cetuximab is limited at about 20% in HNSCC patients with high amplification of EGFR and independent of human papillomavirus (HPV) status. The addition of Cetuximab to platinum-based chemoradiation (CRT) does not lead to an improved outcome. Moreover, the addition of Cetuximab to either carboplatin/paclitaxel chemotherapy or high-dose radiotherapy provided no survival benefit for nonresectable stage III non-small cell lung cancer (NSCLC), which is another cancer subtype that displays high EGFR expression and mutation rates.

EGFR-targeted single agent therapies with either mAb or TKIs result in relatively low response rates and the patients often acquire resistance (Chong et al., 2013; Lim et al., 2018). Less than 5% of HNSCC harbor EGFR mutations, which may explain limited efficacy. Furthermore, multiple extracellular receptors and downstream signaling pathways serve as alternatives, and the persistently activated oncogenic signaling permits cancer resistance to single agent therapy with EGFR-inhibitors.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

In one aspect, the application provides method for treating cancer in a subject. The method may be a combination therapy including at least one antibody. In one embodiment, the method comprises administering to the subject an antibody and a therapeutic agent.

The antibody may be a bispecific antibody. In one embodiment, the antibody has a binding specificity to EGFR and HER3. In one embodiment, the antibody comprises 3 complementary determining regions (CDRs) of SEQ ID NO: 1, 3 CDRs of SEQ ID NO: 2, or 3 CDRs of SEQ ID NO: 4.

In one embodiment, the antibody comprises a heavy chain variable region (VH) having an amino acid sequence with at least at least 70%, 75%, 80%, 85%, 90%, 95%, 98%, 99% sequence identity to SEQ ID NO: 1. In one embodiment, the antibody comprises a heavy chain scFv domain having an amino acid sequence with at least 98% sequence identity to SEQ ID NO: 2. In one embodiment, the antibody comprises a light chain variable region (VL) having an amino acid sequence with at least at least 70%, 75%, 80%, 85%, 90%, 95%, 98%, 99% sequence identity to SEQ ID NO: 4.

In one embodiment, the antibody has a heavy chain and a light chain. In one embodiment, the heavy chain comprises an amino acid sequence with at least at least 70%, 75%, 80%, 85%, 90%, 95%, 98%, 99% sequence identity to SEQ ID NO: 3. In one embodiment, the light chain comprises an amino acid sequence with at least at least 70%, 75%, 80%, 85%, 90%, 95%, 98%, 99% sequence identity to SEQ ID NO: 5.

The therapeutic agent may be any cancer treating agent or combinations of such agents. In one embodiment, the therapeutic agent may be a tyrosine kinase inhibitor (TKI), an alkylating agent, an anti-metabolite, an anti-microtubule agent, a cytotoxic antibiotic, a topoisomerase inhibitor, a chemoprotectant, or a combination thereof. In one embodiment, the therapeutic agent may be Osimertinib, Paclitaxel, Docetaxel, Irinotecan, Carboplatin, Pemetrexed, Cisplatin, or a combination thereof.

In one embodiment, the tyrosine kinase inhibitor (TKI) comprises Erlotinib, Gefitinib, Icotinib, AZD3759, Sapatinib, Afatinib, Dacomitinib, Deratinib, Poziotinib, Tarlox-TKI, Osimertinib, Nazartinib, Olmutinib, Rociletinib, Naquotinib, Lazertinib, EA1045, CLN081, AZ5104, Mobocertinib, its derivative or a combination thereof.

In one embodiment, the method of treating cancer uses a combination treatment including a bispecific antibody and osimertinib. In one embodiment, the antibody may be administered by intravenous drip at least once weekly (QW), bi-weekly or every other week(Q2W), every three weeks (Q3W), or Day 1 and Day 8 every three weeks (D1D8Q3W) in a dosage of, for example, 6 mg/kg, 9 mg/kg, 12 mg/kg, 14 mg/kg, 16 mg/kg, or 21 mg/kg. In one embodiment, if the infusion reaction is tolerable during the first dose 120 min±10 min after the first intravenous drip, the subsequent infusion may be completed within 60-120 min. In one embodiment, the antibody and the therapeutic agent may be used on the same day, and the infusion of the therapeutic agent may be continued after the completion of the antibody infusion. In one embodiment, Osimertinib is administered at a dose of at least about 40 mg/kg, about 80 mg/kg, about 120 mg/kg, about 160 mg/kg or about 180 mg/kg.

In one embodiment, the alkylating agent comprises Busulfan, Cyclophosphamide, Temozolomide, Carboplatin, Cisplatin, or a combination thereof.

In one embodiment, the method uses a combination including a bispecific antibody and Carboplatin. In one embodiment, Carboplatin is administered at a dose from about 100 mg/m2 to about 500 mg/m2. In one embodiment, Carboplatin is administered at a dose of at least about 200 mg/m2, about 250 mg/m2, about 300 mg/m2, about 360 mg/m2, about 400 mg/m2, or about AUC 5 mg/ml/min, about AUC 6 mg/ml/min, about AUC 7 mg/ml/min. In one embodiment, carboplatin is administered at a dose at about AUC 5 mg/ml/min.

In one embodiment, the method uses a combination including a bispecific antibody and Cisplatin. In one embodiment, cisplatin is administered at a dose from about 10 mg/m2 to about 180 mg/m2. In one embodiment, Cisplatin is administered at a dose of at least about 15 mg/m2, about 20 mg/m2, about 30 mg/m2, about 50 mg/m2, about 75 mg/m2, about 100 mg/m2, or about 120 mg/m. In one embodiment, cisplatin is administered at a dose of 100 mg/m2, Q3W. In one embodiment, the method further comprises step of determining the does toxicity after initial infusion or first course of treatment. If the does is too toxic, the dose may be reduced to 80%.

In one embodiment, the anti-metabolite may include 6-mercaptopurine, Fludarabine, 5-fluorouracil, Gemcitabine, Cytarabine, Pemetrexed, Methotrexate, its derivative or a combination thereof.

In one embodiment, the method uses a combination of a bispecific antibody and Pemetrexed. In one embodiment, pemetrexed is administered at a dose from about 150 mg/m2 to about 800 mg/m2. In one embodiment, Pemetrexed is administered at a dose of at least about 250 mg/m2, about 500 mg/m2, or about 750 mg/m2. In one embodiment, Pemetrexed is administered at a dose of about 500 mg/m2. In one embodiment, the method further comprises step of determining the does toxicity after initial infusion or first course of treatment. If the does is too toxic, the dose is reduced to 80%.

In one embodiment, the application uses a combination therapy including a bispecific antibody and a combination of pemetrexed and cisplatin. In one embodiment, the antibody may be administered by intravenous drip at least once weekly (QW). In one embodiment, the administration of pemetrexed and cisplatin (AP) may follow the drug instructions and standard usage and may be administered immediately after the antibody is completed.

In one embodiment, the anti-microtubule agent comprises Docetaxel, Eribulin, Ixabepilone, Paclitaxel, Vinblastine, its derivative, or a combination thereof.

In one embodiment, the method uses a combination treatment including a bispecific antibody and Paclitaxel. In one embodiment, the antibody may be administered by intravenous drip once a week (QW). In one embodiment, Paclitaxel may be administered at a dose from about 20 mg/m2 to about 200 mg/m2. In one embodiment, Paclitaxel is administered at a dose of at least about 40 mg/m2, about 80 mg/m2, about 135 mg/m2, or about 175 mg/m2. In one embodiment, the dosage of paclitaxel may be 80 mg/m2 QW.

In one embodiment, the antibody and paclitaxel may be used on the same day. In one embodiment, after the antibody infusion, paclitaxel may be pretreated and injected within 3 hours. In one embodiment, the method may further comprise step of determining the does toxicity after initial infusion. If the does is too toxic, the dose is reduced to 80%.

In one embodiment, the application provides a method of treating cancer using a combination therapy including a bispecific antibody and a combination of paclitaxel and cisplatin. In one embodiment, antibody may be administered by intravenous drip at least once weekly (QW). In one embodiment, the administration of paclitaxel and cisplatin (TP) may follow the drug instructions and standard usage and may be administered immediately after the antibody is completed.

In one embodiment, the method may use a combination of a bispecific antibody and docetaxel. In one embodiment, docetaxel is administered at a dose of at least about 35 mg/m2 D1D8D215Q3W. In one embodiment, the method further comprises step of determining the does toxicity after initial infusion. If the does is too toxic, the dose is reduced to 80%.

In one embodiment, the cytotoxic antibiotics comprises Dactinomycin, Bleomycin, Daunorubicin, Doxorubicin, its derivative, or a combination thereof.

In one embodiment, the topoisomerase inhibitor comprises Etoposide, Irinotecan, Topotecan, its derivative or a combination thereof.

In one embodiment, the method may use a combination of a bispecific antibody and irinotecan. In one embodiment, the antibody is administered by intravenous drip every 2 weeks (Q2W). In one embodiment, irinotecan may be administered at a dose from about 50 mg/m2 to about 250 mg/m2. In one embodiment, irinotecan may be administered at a dose of at least about 80 mg/m2, 130 mg/m2, 150 mg/m2, 180 mg/m2, 200 mg/m2, or 220 mg/m2. In one embodiment, the dose of irinotecan may be 180 mg/m2 Q2W, and the infusion method may follow the drug instructions. In one embodiment, the antibody and irinotecan may be used on the same day, and irinotecan may be injected after the antibody infusion.

In one embodiment, the chemoprotectant comprises Leucovorin or its derivative thereof.

In one embodiment, the antibody may be combined with one or more therapeutic agent in treating cancer. In one embodiment, the antibody may be combined with a standard chemotherapy combination. In one embodiment, the therapeutic agent or combination of therapeutic agents are administered according to the established dosage, regime, or methodology in cancer therapy.

The antibody and the therapeutic agent may be administered simultaneously or sequentially as one treatment session.

In one embodiment, the antibody and the therapeutic agent may be separately administered to the subject in alternating treatment session. In one embodiment, the antibody and the therapeutic agent are administered simultaneously and sequentially. In one embodiment, the antibody is administered at a separate time from the therapeutic agent.

In one embodiment, the antibody may be administered in a first treatment session and the therapeutic agent is administered in a second treatment session. In one embodiment, the duration of the first treatment session may be from about 7 days to about 728 days. In one embodiment, the duration of the second treatment session is from about 1 day to about 728 days. In one embodiment, the gap between the first treatment session and the second treatment session is from about 7 to about 21 days.

In one embodiment, the antibody may be administered once a week (Q1W), every two weeks (Q2W), every three weeks (Q3W), or Day 1 and Day 8 every three weeks (D1D8Q3W).

The antibody may be administered at a fixed dose, a dose by mg/kg, or a dose by mg/m2. In one embodiment, the antibody is administered at a dose from about 0.1 mg/kg to about 50 mg/kg. In one embodiment, the antibody is administered at a dose of at least about 0.3 mg/kg, about 1.2 mg/kg, about 3.0 mg/kg, about 6.0 mg/kg, about 9.0 mg/kg, about 12.0 mg/kg, about 16.0 mg/kg, about 21.0 mg/kg or about 28.0 mg/kg.

The therapeutic agent may be administrated following the drug instruction and standard usage or dosage regime. In one embodiment, the therapeutic agent may be administered at a dose from about 6.0 mg/Kg to about 28.0 mg/Kg.

In one aspect, the application provides therapeutic composition for treating a subject with cancer. The therapeutic composition may include a combination of an antibody and a therapeutic agent as disclosed herein.

The antibody may be a bispecific antibody having a binding specificity to EGFR and HER3 bispecific. In one embodiment, the antibody comprises 3 complementary determining regions (CDRs) of SEQ ID NO: 1, 3 CDRs of SEQ ID NO: 2, or 3 CDRs of SEQ ID NO: 4.

In one embodiment, therapeutic agent may be any therapeutic agent or combinations disclosed herein including, for example, Osimertinib, Carboplatin, Cisplatin, Pemetrexed, Paclitaxel, its derivative, or a combination thereof.

In one embodiment, both or either of the antibody and the therapeutic agent are in the form of a pharmaceutical formulation to be administered simultaneously, sequentially, or concurrently.

In one aspect, the application further provides a kit comprising a first container, a second container and a package insert. The first container comprises at least one dose of a first therapeutic composition comprising an antibody, the second container comprises at least one dose of a second therapeutic composition comprising a therapeutic agent, and the package insert comprises instructions for treating a subject for cancer using the first and the second therapeutic compositions.

In one embodiment, the instructions may state that the first and the second therapeutic compositions are intended for use in treating a subject having a cancer that tests positive for EGFR expression.

The cancer may be a solid tumor. In one embodiment, the cancer is lung adenocarcinoma, head/neck squamous cell cancer, rectal cancer, colon cancer, squamous cell lung cancer, thyroid cancer, bladder cancer, melanoma, cervical cancer, prostate cancer, breast cancer, uterine/endometrial cancer, pancreatic cancer, ovarian cancer, or papillary kidney cancer

In one embodiment, the cancer comprises a solid tumor that tests positive for EGFR expression and is selected from the group consisting of: lung adenocarcinoma, head/neck squamous cell cancer, rectal cancer, colon cancer, squamous cell lung cancer, thyroid cancer, bladder cancer, melanoma, cervical cancer, prostate cancer, breast cancer, uterine/endometrial cancer, pancreatic cancer, ovarian cancer, and papillary kidney cancer.

In one embodiment, the cancer is an advanced or metastatic solid tumor.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 demonstrates the effect of a representative bispecific antibody, SI-B001, and Osimertinib (Osi)-based combination therapy when treating HCC827-936 tumor cell xenografts in nude mice, where the tumor volumes were measured as means of each treatment (3 dose regimens) and control group with standard errors indicated (1a); the curves of body weight during the treatment were shown (1b); and the expression of EGFR and HER3 in HCC827-936 cells derived tumors was detected by Flow Cytometric Fluorescence Sorting (FACS)(1c);

FIG. 2 demonstrates the effect of SI-B001 and Osimertinib (Osi)-based combination therapy when treating NCI-H1975 tumor cell xenografts in nude mice, where the tumor volumes were measured as means of each treatment (3 dose regimens) and control group with standard errors indicated (2a); the curves of body weight during the treatment were shown (2b); and the expression of EGFR and HER3 in NCI-H1975 cells derived tumors was detected by FACS(2c);

FIG. 3 demonstrates the effect of SI-B001 and CarboTaxol (Paclitaxel and Carboplatin)-based combination therapy when treating Fadu tumor cell xenografts in nude mice, where the tumor volumes were measured as means of each treatment (3 dose regimens) and control group with standard errors indicated (3a); the curves of body weight during the treatment were shown (3b); and the expression of EGFR and HER3 in Fadu cells derived tumors was detected by FACS(3c);

FIG. 4 shows the measurement of tumor volume and the percentage of tumor growth inhibition of SI-B001+Chemo when compared to (4a) either SI-B001 or Chemo alone, (4b) Cetuximab+Chemo, and (4c) either SI-B001 alone, Cetuximab alone, or Cetuximab+Chemo;

FIG. 5 demonstrates the effect of SI-B001 and Cisplatin/Pemetrexed-based combination therapy when treating HCC827 tumor cell xenografts in nude mice, where the tumor volumes were measured as means of each treatment (3 dose regimens) and control group with standard errors indicated (5a); curves of body weight during the treatment were shown (5b);

FIG. 6 shows the comparative advantage of SI-B100, TKI, and chemotherapeutics in combination therapy for treating human tumor mouse xenograft models as measured tumor growth rates affected by (6a) Cetuximab and Osimertinib, alone or combined; (6b) SI-B001 low and Osimertinib, alone or combined; (6c) SI-B001 mid and Osimertinib, alone or combined; (6d) SI-B001 high and Osimertinib, alone or combined; (6e) Cetuximab and Chemo, alone or combined; (6f) SI-B001 low and Chemo, alone or combined; (6g) SI-B001 mid and Chemo, alone or combined; and (6h) SI-B001 high and Chemo, alone or combined; and

FIG. 7 shows the water fall plot of the tumor responses in patients who received (7a) SI-B001 plus AP/TP or Docetaxel; (7b) SI-B001 plus Paclitaxel; and (7c) SI-B001 plus Irinotecan.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, compositions, and kits for treating cancers using a combination therapy including an antibody and an additional therapeutic agent.

### Anti-EGFR/HER3 Antibody Therapy (SI-B001)

SI-B001 is a bispecific tetravalent anti-EGFR/HER3 monoclonal antibody (also known as SI-1X6.4 in U.S. Pat. No. 10,919,977B2, hereby incorporated by reference in its entirety). Upon administration, SI-B001 simultaneously binds to EGFR and HER3 on cancer cells, thereby, preventing receptor phosphorylation and oncogenic signaling. In essence, SI-B001 embodies the binding to both EGFR and HER3 in a single therapeutic antibody.

Antibody-based immune therapy inhibits cancer cell proliferation by means of blocking the binding of growth factors to the receptor and preventing homo- and heterodimer signaling states on the cell surface, and by means of promoting internalization and degradation. On the other hand, small molecules, such as tyrosine kinase inhibitors (TKI), inhibits the cytoplasmic kinase activity induced by the oncogenic signaling from one or more EGFR family members. All solid tumor cells are heterogenous in nature, namely, even the status of EGFR mutations and aberrant expression in a cancer of the same patient may vary from time to time. The combination therapy combining the antibodies disclosed herein and additional therapeutic agents provides significant technical advantage of increasing the efficacy of treating heterogenous tumor cells at different stages of tumor progression, decreasing the incidence of relapse, or both. In one embodiment, SI-B001 in combination with TKIs combines antibody-based inhibition of EGFR family member(s)-mediated oncogenic signaling with the inhibition of cytoplasmic signaling by EGFR TKI by attacking the vertical pathway of EGFR oncogenic signaling.

### SI-B001 Combination Therapy

In some embodiments, the disclosure provides a method of treating cancer in a patient in need thereof. The cancer may be, without limitation, solid tumors, soft tissue sarcoma, squamous cell carcinoma, head and neck squamous cell carcinoma (HNSCC), non-small-cell lung carcinoma (NSCLC), esophageal squamous cell carcinoma (ESCC), or EGFR-expressing cancer. The method may include administering to the patient an effective amount of SI-B001 optionally in combination with one or more standard of care treatments, additional therapeutic agents, or a combination thereof.

Standard of care treatments for cancer, such as but not limited to HNSCC, NSCLC, and ESCC, are well known to one of ordinary skill in the art and include surgery, radiotherapy, chemotherapy, photodynamic therapy, target therapy, or targeted immunotherapy, or a combination thereof. In some embodiments, the standard of care treatments is selected from chemotherapy using carboplatin (PARAPLATIN®, BMS), cisplatin (PLANTINOL®, BMS), docetaxel (TAXOTERE®, Sanofi-Aventis), irinotecan (CAMPTOSAR®, Pfizer), pemetrexed disodium (ALIMTA®, Eli Lilly), afatinib dimaleate, alectinib (ALECENZA®, Genentech), bleomycin, brigantinib, ceritinib (ZYKADIA®, Novartis), crizotinib (XALKORI®, Pfizer), dabrafenib, doxorubicin HCl, etoposide, everolimus, 5-fluorouracil, gemcitabine HCl, hydroxyurea, mechlorethamine HCl, methotrexate, sunitinib, trametinib, vinorelbine tartrate (NAVELBINE®, Pierre Fabre), topotecan HCl, a derivative or a combination thereof.

In some embodiments, the additional therapeutic agent may be a tyrosine kinase inhibitor fortarget chemotherapy, including, forexample, erlotinib, gefitinib, icotinib, AZD3759, sapatinib, afatinib, dacomitinib, deratinib, poziotinib, nazartinib, olmutinib, rociletinib, naquotinib, lazertinib, EA1045, CLN081, AZ5104, mobocertinib, or a derivative thereof. In some embodiments, the additional therapeutic agent is Osimertinib (TAGRISSO®, AstraZeneca), a third-generation irreversible EGFR inhibitor designed to target mutant EGFR as well as aberrantly expressed EGFR without affecting wild type EGFR. Osimertinib is well tolerated in patients with advanced or metastatic NSCLC.

In some embodiments, the additional therapeutic agent may be a monoclonal antibody for targeted immunotherapy, including for example, cetuximab (anti-EGFR antibody, ERBITUX®, Lilly), nivolumab (anti-PD1 antibody, OPDIVO®, BMS), pembrolizumab (anti-PD1 antibody, KEYTRUDA®, Merck), cemiplimab (anti-PD1 antibody, LIBTAYO®, Regeneron), atezolizumab (anti-PD-L1 antibody, TECENTRIQ®, Roche), durvalumab (anti-PD-L1 antibody, IMFINZI®, AstraZeneca), bevacizumab (anti-VEGF antibody, AVASTIN®, Roche), or a biosimilar thereof.

In some embodiments, SI-B001 is administered to a patient as a first-line monotherapy for HNSCC, NSCLC, and ESCC. In other embodiments, SI-B001 is administered to a patient as a first-line treatment in combination with a standard of care treatment for HNSCC, NSCLC, and ESCC, including surgery, radiotherapy, chemotherapy, photodynamic therapy, or targeted immunotherapy, or a combination thereof.

In some embodiments, SI-B001 is administered to a patient with lung cancer as a first-line treatment in combination with a standard of care chemotherapy or docetaxel (TAXOTERE®, Sanofi-Aventis), including NSCLC but not limited to, recurrent and metastatic non-small cell lung cancer. In other examples, SI-B001 is administered to a patient with head and neck cancer as a first-line treatment in combination with a standard of care chemotherapy or paclitaxel (TAXOL®, BMS; ABRAXANE®, Abraxis), including HNSCC but not limited to, recurrent and metastatic HNSCC. In another examples, SI-B001 is administered to a patient with esophageal cancer as a first-line treatment in combination with a standard of care chemotherapy or Irinotecan (CAMPTOSAR®, Pfizer), including ESCC but not limited to, relapsed and metastatic ESCC.

In some embodiments, when a standard of care treatment fails, such as when surgery fails to remove all cancerous tissue or the cancer is partially resistant to a chemotherapy or an immunotherapy, a second-line treatment is used that can include a well-known second-line treatment to treat HNSCC, NSCLC, and ESCC. Accordingly, in some embodiments, the disclosure provides a method of treating HNSCC, NSCLC, and ESCC in a patient wherein the cancer is resistant to a first-line therapy, said method comprising administering SI-B001 optionally in combination with a second-line treatment.

In some embodiments, the disclosure provides a method of treating cancer comprising administering SI-B001 as the second-line treatment. In some embodiments, the disclosure provides a method of treating a resistant cancer comprising administering SI-B001 in combination with another second-line treatment or standard of care second-line treatment for HNSCC (ClinicalTrials.gov ID: NCT05054439, S206), NSCLC (ClinicalTrials.gov ID: NCT05020457, S201), and ESCC (ClinicalTrials.gov ID: NCT05022654, S207, incorporated hereby by reference in its entirety). In some embodiments, the second-line treatment may be a chemotherapy. For example, SI-B001 is administered to a patient with HNSCC, NSCLC, and ESCC as a second-line treatment in combination with a standard of care chemotherapy or paclitaxel in the treatment of HNSCC, NSCLC, and ESCC, including but not limited to, recurrent, relapsed, and/or metastatic HNSCC, NSCLC, and ESCC. In some embodiments, the second-line treatment may be a target therapy. For example, SI-B001 is administered to a patient with HNSCC, NSCLC, and ESCC as a second-line treatment in combination with a standard of care chemotherapy or Osimertinib in the treatment of cancer, such as HNSCC, NSCLC, and ESCC, including but not limited to, recurrent, relapsed, and/or metastatic HNSCC, NSCLC, and ESCC.

In some embodiments when the first-line or second-line standard of care treatment fails, such as when chemotherapy continues to fail and remission occurs, a third-line treatment is administered to the patient. In some embodiments, the disclosure provides a method of treating HNSCC (ClinicalTrials.gov ID: NCT05054439, S206), NSCLC (ClinicalTrials.gov ID: NCT05020457, S201), and ESCC (ClinicalTrials.gov ID: NCT05022654, S207) resistant to both first-line therapy and second-line therapy comprising administering SI-B001 as the third-line treatment. In some embodiments, the disclosure provides a method of treating HNSCC, NSCLC, and ESCC resistant to both first-line therapy and second-line therapy comprising administering SI-B001 in combination with another third-line treatment or standard of care third-line treatment for HNSCC, NSCLC, and ESCC.

In some embodiments, SI-B001 is administered as a sensitizer for the treatment of HNSCC, NSCLC, and ESCC in a patient in need thereof. Without being bound by any theory, it is believed that SI-B001 increases the efficacy of the standard of care, first-line, second-line, or third-line treatments for HNSCC, NSCLC, and ESCC. In some embodiments, the disclosure provides a method of treating HNSCC, NSCLC, and ESCC in a patient in need thereof, comprising administering SI-B001 to the patient prior to administration of one or more of standard of care, first-line, second-line, or third-line treatment. In some embodiments, administration of SI-B001 results in a more effective treatment of HNSCC, NSCLC, and ESCC compared to treatment in the absence of administration of SI-B001. In some embodiments, the disclosure provides a method of treating HNSCC, NSCLC, and ESCC in a patient in need thereof, comprising administering SI-B001 to the patient after administration of one or more of standard of care, first-line, second-line, or third-line treatment.

One of ordinary skill in the art will understand the amount and dosing regimen to administer such additional therapeutic agents for the treatment of HNSCC, NSCLC, and ESCC. By way of example, the administration of exemplary therapeutic agents suitable for treating HNSCC, NSCLC, and ESCC is summarized in Table 1.

The term “pharmaceutical formulation” refers to a process in which different chemical substances, including the active pharmaceutical compound that is both stable and acceptable to the patient, are combined to produce a final medicinal product in a dosage form. For orally administered drug, the active pharmaceutical compound is incorporated into a non-toxic carrier, adjuvant, or vehicle that does not destroy the pharmacological activity of the compound with which it is formulated. The formulated pharmaceutical compound is compatible with these other substances in a way that does not cause harm, whether direct or indirect. The pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the disclosure may be administered orally, parenterally, by inhalation spray, topically (as by powders, ointments, or drops), rectally, nasally, buccally, intravaginally, intracisternally, or via an implanted reservoir. The term “parenteral” as used herein includes subcutaneous, intravenous, intramuscular, intraarticular, intra-synovial, intrasternal, intrathecal, intrahepatic, intralesional, and intracranial injection or infusion techniques. Preferably, the compositions are administered orally, intraperitoneally, or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium.

It should also be understood that a specific dosage and treatment regimen for any patient will depend upon a variety of factors, including the activity of the specific compound employed, the age, body weight, general health, sex, diet, time of administration, rate of excretion, drug combination, and the judgment of the treating physician and the severity of the disease being treated. The amount of a compound of the disclosure in the composition will also depend upon the compound in the composition.

The compounds and compositions, according to the method of the present disclosure, may be administered using any amount and any route of administration effective for treating a cancer, such as those disclosed herein. The exact amount required may vary from subject to subject, depending on the species, age, and general condition of the subject, the severity of the cancer, the agent, its mode of administration, and the like. Compounds and compositions of the present disclosure are preferably formulated in a uniformity of dosage for ease of administration of a physically discrete unit of agent appropriate for the patient to be treated. It will be understood, however, that the total daily usage of the compounds and compositions of the present disclosure will be decided by the attending medical professionals within the scope of professional medical judgment. The specific effective dose level for any particular patient or organism will depend upon a variety of factors including the cancer being treated and the severity of the cancer; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Injectable preparations, such as sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, and isotonic sodium chloride solution. Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

The kit of the present disclosure is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

The terms “a”, “an” and “the” as used herein are defined to mean “one or more” and include the plural unless the context is inappropriate.

The terms “polypeptide”, “peptide”, and “protein”, as used herein, are interchangeable and are defined to mean a biomolecule composed of amino acids linked by a peptide bond.

The term “antigen”, “antigenic site”, and “epitope” refers to an entity or fragment thereof which can induce an immune response in an organism, particularly an animal, more particularly a mammal including a human. The term includes immunogens and regions thereof responsible for antigenicity or antigenic determinants.

The term “antibody” is used in the broadest sense and specifically covers single monoclonal antibodies (including agonist and antagonist antibodies), antibody compositions with polyepitopic specificity, as well as antibody fragments (e.g., Fab, F(ab′)₂, and Fv), so long as they exhibit the desired biological activity. In some embodiments, the antibody may be monoclonal, polyclonal, chimeric, single chain, bispecific or bi-effective, human and humanized antibodies as well as active fragments thereof. Examples of active fragments of molecules that bind to known antigens include Fab, F(ab′)₂, scFv and Fv fragments, including the products of a Fab immunoglobulin expression library and epitope-binding fragments of any of the antibodies and fragments mentioned above. In some embodiments, antibody may include immunoglobulin molecules and immunologically active portions of immunoglobulin molecules, i.e. molecules that contain a binding site that can bind to an antigen, an antigenic site, or an epitope. The immunoglobulin can be of any type (IgG, IgM, IgD, IgE, IgA and IgY) or class (IgG1, IgG2, IgG3, IgG4, IgA1 and IgA2) or subclasses of immunoglobulin molecule. In one embodiment, the antibody may be whole antibodies and any antigen-binding fragment derived from the whole antibodies.

A typical antibody refers to heterotetrameric protein comprising typically of two heavy (H) chains and two light (L) chains. Each heavy chain is comprised of a heavy chain variable domain (abbreviated as VH) and a heavy chain constant domain. Each light chain is comprised of a light chain variable domain (abbreviated as VL) and a light chain constant domain. The “light chains” of antibodies (immunoglobulins) from any vertebrate species can be assigned to one of two clearly distinct types, called kappa (κ) and lambda (λ), based on the amino acid sequences of their constant domains. The VH and VL regions can be further subdivided into domains of hypervariable complementarity determining regions (CDR), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interacts with the antigen.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against one antigenic site as a monoclonal monospecific antibody, or more than one antigenic site as a monoclonal multi-specific antibody. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any method. For example, the monoclonal antibodies to be used in accordance with the disclosure may be made by the hybridoma method first described by Kohler & Milstein, Nature, 256:495 (1975), or may be made by recombinant DNA methods (see, e.g., U.S. Pat. No. 4,816,567).

The monoclonal antibodies may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 [1984]). Monoclonal antibodies can be produced using various methods including mouse hybridoma or phage display (see Siegel. Transfus. Clin. Biol. 9:15-22 (2002) for a review) or from molecular cloning of antibodies directly from primary B cells (see Tiller. New Biotechnol. 28:453-7 (2011)). In the disclosure antibodies were created by methods of immunizing rabbits, mice, or llama in combination with subsequent strategies like hybridoma or display. Rabbits are known to create antibodies of high affinity, diversity and specificity (Weber et al. Exp. Mol. Med. 49:e305). Besides immunization of rabbits followed by B cell culture, other common strategies for antibody generation and discovery include immunization of other animals (e.g., mice, llamas) followed by hybridoma and/or display on phage, yeast, or mammalian cells; or display using synthetic variable gene libraries. This general method of antibody discovery is similar to that described in Seeber et al. PLOS One. 9:e86184 (2014).

The term “antigen-binding or epitope-binding portion or fragment” refers to fragments of an antibody that are capable of binding to an antigen. These fragments may be capable of the antigen-binding function and additional functions of the intact antibody. Examples of binding fragments include, but are not limited to, a single-chain Fv fragment (scFv) consisting of the VL and VH domains of a single arm of an antibody connected in a single polypeptide chain by a synthetic linker or a Fab fragment which is a monovalent fragment consisting of the VL, constant light (CL), VH and constant heavy 1 (CH1) domain. Antibody fragments can be even smaller sub-fragments and can consist of domains as small as a single CDR domain, the CDR3 regions from either the VL and/or VH domains (for example see Beiboer et al., J. Mol. Biol. 296:833-49 (2000)). Antibody fragments are produced using conventional methods known to those skilled in the art. The antibody fragments can be screened for utility using the same techniques employed with intact antibodies.

The “antigen- or epitope-binding fragments” can be derived from an antibody of the disclosure by several known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)₂ fragment that has two antigen combining sites and is still capable of cross-linking antigen. The Fab fragment may contain the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other, chemical couplings of antibody fragments are also known.

“Fv” is the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called α, delta, epsilon, γ, and μ, respectively. The subunit structures and three-dimensional configurations of different classes of immunoglobulins are well known.

“Multivalent” antibodies bind to multiple sites on one target, which may result in higher functional affinity and avidity over their monomeric forms depending on the number of binding sites. All antibodies are multivalent e.g., IgGs are bivalent and and IgMs are decavalent.

“Homology” between two sequences is determined by sequence identity. If two sequences which are to be compared with each other differ in length, sequence identity preferably relates to the percentage of the nucleotide residues of the shorter sequence which are identical with the nucleotide residues of the longer sequence. Sequence identity can be determined conventionally with the use of computer programs. The deviations appearing in the comparison between a given sequence and the above-described sequences of the disclosure may be caused for instance by addition, deletion, substitution, insertion, or recombination.

The term “targeted chemotherapy” refers to molecularly targeted therapy for treating cancer, as to other medical treatments, such as pharmacotherapy, hormonal therapy, and cytotoxic chemotherapy. The first-in-class drug of target therapy or targeted chemotherapy is Imatinib (GLEEVEC®, Novartis), an anti-cancer tyrosine kinase inhibitor (TKI). This class of TKIs have substantially improved outcomes in chronic myelogenous leukemia, acute lymphocytic leukemia that are Philadelphia chromosome-positive, certain types of gastrointestinal stromal tumors, hypereosinophilic syndrome, chronic eosinophilic leukemia, systemic mastocytosis, myelodysplastic syndrome, and dermatofibrosarcoma protuberans. TKIs have also been used to treat other diseases, such as idiopathic pulmonary fibrosis.

The term “EGFR-TKI” refers to TKI drugs that inhibits tyrosine kinase activities of EGFR signalling pathway. Tyrosine kinases are enzymes responsible for the activation of many proteins by signal transduction cascades, such as the EGFR family member-mediated signalling. TKIs are also known as “tyrosine phosphorylation inhibitor”, which do not inhibit protein kinases that phosphorylate serine or threonine residues and can discriminate between the kinase domains of the EGFR and that of the insulin receptor. The proteins are activated by adding a phosphate group to the protein (phosphorylation). Three generations of EGFR-TKIs have been developed as targeted chemotherapy for treating cancer harbouring EGFR mutations, which are classified based on the mechanism of action and the clinical benefits, (Sullivan and Planchard, 2017)

The term “Bliss independence score” refers to the widely used Bliss independence model for analysing drug combination data when screening for candidate drug combinations. The method compares the observed combination response (Y_{O}) with the predicted combination response (Y_{P}), which is obtained based on the assumption that there is no effect from drug-drug interactions. Suppose two drugs, A and B, both inhibit tumour growth: drug A at dose a inhibits Yₐ percent of tumor growth and drug B at dose b inhibits Y_{b} percent of tumour growth. If two drugs work independently, the combined percentage inhibition Y_{ab,P} can be predicted using the complete additivity of probability theory as:

Y_{ab,P}=Yₐ+Y_{b}−Yₐ×Y_{b}

The disclosure may be understood more readily by reference to the following detailed description of specific embodiments and examples included herein. Although the disclosure has been described with reference to specific details of certain embodiments thereof, it is not intended that such details should be regarded as limitations upon the scope of the disclosure. Indeed, various modifications of the disclosure in addition to those described herein will become apparent to those skilled in the art from the foregoing description and accompanying drawings. Such modifications are intended to fall within the scope of the appended claims.

### EXAMPLES

### Example 1. Xenograft Models of Human Cancer

Xenotransplantation of human cancer cells into immunocompromised mice is a gold-standard model used for pre-clinical oncology research and testing of anti-cancer therapies. In general, human cancer cell lines are injected below the skin of the mouse. A drug or drug combination is administered into one or more groups of mice while the control group does not receive drug. The primary outcome parameter is growth inhibition by the drug, for which tumor size is measured over time, often externally with calipers. After the tumor reaches a certain size, the mice are euthanized. Drug effects on the tumor are further analyzed on a tissue level or molecular level (e.g., DNA, proteins). The primary outcome parameter of a xenograft model for testing drug efficacy is the growth of the treated tumor versus control. Typically, xenograft tumors grow from 100 mm³ (6 mm diameter) to 1000 mm³ (12 mm diameter) in several weeks. The growth rate often declines with larger tumor sizes, as opposed to exponential growth in (2D) in vitro experiments. Tumor growth inhibition varies per drug and implanted cell line. The effect of therapeutics can be directly on tumor cells, or indirectly via the microenvironment such as the inhibition of vessel formation, or both.

Xenograft models of HNSCC and NSCLC were used to evaluate the safety and therapeutic effect of SI-B001 monotherapy and in combination with standard care, chemotherapy, and/or TKI-based target therapy. The experiment animals used were of Balb/c-nu mouse, female, ages 5^{˜}7 weeks, weight 17^{˜}21 g.

To establish xenograft model of HNSCC, human head and neck cancer cells, FaDu cells, were transplanted into the mice to establish the xenograft model of HNSCC. FaDu is a cell line isolated from a hypopharyngeal tumor of a squamous cell carcinoma patient, which expresses wild type EGFR (ATCC).

To establish xenograft model of NSCLC, two lung cancer cell lines expressing were used. Human lung adenocarcinoma cell line HCC827_936 was clonally derived from HCC827 cell line isolated from NSCLC tissue of a patient (ATCC). HCC827_936 cells express EGFR mutations of exon19del and exon20ins. The other lung cancer cell line, NCI-H1975 cells (ATCC), express the EGFR L858R/T790M double mutation.

Each of the three human cancer cell lines was cultured in RPMI1640 medium containing 10% fetal bovine serum, collected in the exponential growth phase, resuspended in PBS to a suitable concentration, and used for subcutaneous tumor implantation in mice.

### Example 2. SI-B001 and Osimertinib Combination Therapy in HCC827 936 Model

To evaluate the anti-tumor efficacy and safety of the experimental drugs SI-B001 in combination with Osimertinib (Osi), 40 female nude mice were subcutaneously implanted with 5×10⁶ HCC827_936 cells and the cells were resuspended in PBS (0.1 ml/mouse). When the tumor grew to an average volume of about 163 mm³, randomly grouped according to tumor size and mouse weight to establish HCC827_936 xenograft model.

Saline and SI-B001 were administered by intravenous infusion once a week. SI-B001 was given at 3 different dose regimens, 9 mg/kg, 16 mg/kg, and 28 mg/kg. DMSO and Osimertinib were administered were given orally once a week. Osimertinib was given at a fixed dose regimen, 96 mg/kg (Table 2).

Tumor volume (mm³) V=½×(a×b)², where a represents the long diameter and b represents the short diameter. Relative tumor volume (RTV), RTV=Vt/V0, where V0 is the tumor volume at time 0 and Vt is the tumor volume after treatment at time t. The T/C value (%) is an indicator of the tumor response to treatment and is the most used evaluation indicator. T/C %=T_{RTV}/C_{RTV}×100%, where T_{RTV} is treatment group average RTV and C_{RTV} is control group average RTV. The T/C value (%) can also be calculated as T/C %=T_{TW}/C_{TW}×100%, where T_{TW} is the average tumor weight at the end of the treatment group experiment and C_{TW} is the average tumor weight at the end of the control group experiment. The relative tumor inhibition rate (Tumor growth inhibition, TGI) is one of the indicators of the tumor response to treatment, TGI %=100%−T/C %.

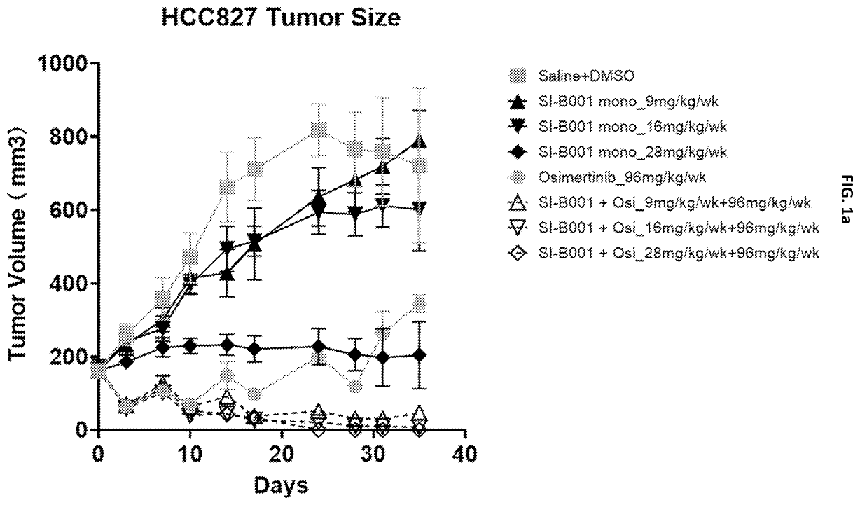

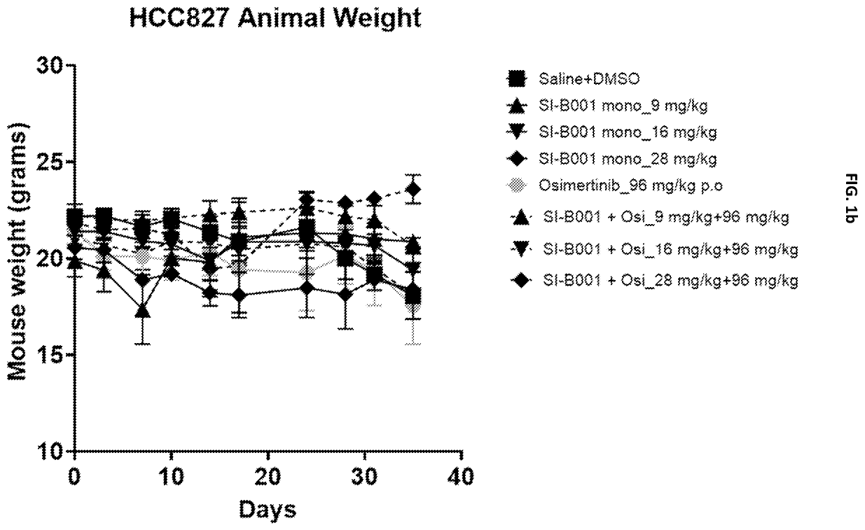

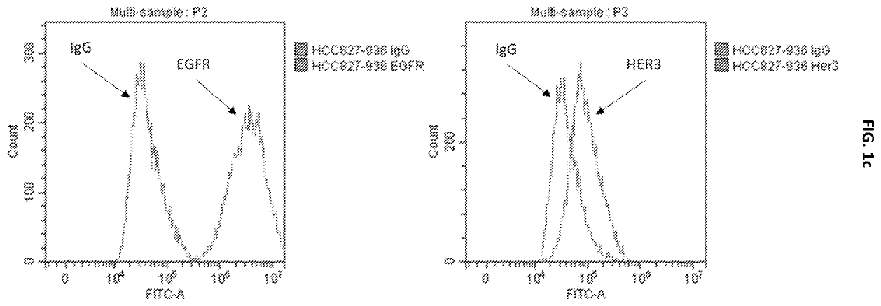

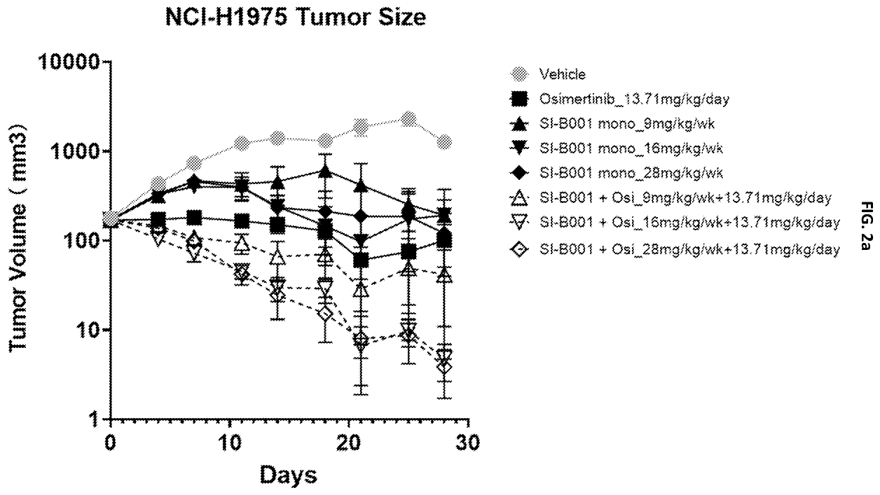

The average tumor volume of mice in the vehicle control group was 720.68 mm³ on day 31, the average tumor volume of the control Osimertinib single-agent group was 344.53 mm³ on day 31, and TGI (%) was 54.89% (p=0.262). The average tumor volume on day 31 of SI-B001 single-agent, dose group 9/16/28 mg/kg/mouse, was 789.65 mm3, 601.42 mm3, and 203.92 mm³ respectively, and TGI (%) was −10.71%, 24.64% and 73.20% respectively (compared to the vehicle control group, p=0.783, p=0.654, p=0.123). The combination of SI-B001 and Osimertinib in each dose group (9/16/28+96 mg/kg/mouse) corresponds to the average tumor volume on day 31 of 46.79 mm³, 7.75 mm³, 0 mm³, TGI % were 93.74%, 99.07%, and 100.00% (compared to the single-agent SI-B001 at the corresponding dose level, p=0.002, p=0.012, p=0.268; compared to the single-agent Osimertinib, p=0.007, p=0.036, p=0.044), the anti-tumor effect is better than the corresponding single-agent SI-B001 and single-agent Osimertinib. In summary, all dose group of SI-B001 combined with Osimertinib have a significant higher anti-tumor effect (FIG. 1a, Table 3). The anti-tumor effect is better than the corresponding single-agent SI-B001 and single-agent Osimertinib, showing a significant synergistic anti-tumor effect with the combination therapy. And the level of tumor inhibition is positively associated with increased dose of SI-B001.

The weight of mice was measured across the experiment (FIG. 1b). In the vehicle group #1, 2 mice died before D31, the mortality rate was 40%. In SI-B001 single-agent low dose groups #3, 1 mouse died before D31, the mortality rate was 20%. In SI-B001 single-agent middle dose groups #4, 3 mouse died before D31, the mortality rate was 60%. In SI-B001 single-agent high dose groups #5, 3 mice died before D31, the mortality rate was 60%. In the Osimertinib single-agent group #2, 3 mice died before D31, the mortality rate was 60%. In the combination group #6, SI-B001 low dose, no mice died before D31. The other two combination groups #7 and #8, 3 mice died before D31, the mortality rate was 60%. There is no clear trend among the groups in terms of number of deaths. This showed no obvious toxicity when combining SI-B001 and Osimertinib.

To verify tumor tissue targets in tumor-bearing mice at the end of the experiment, the tumor tissues were taken after the mice were euthanized, and the Flow Cytometric Fluorescence Sorting (FACS) analysis was used to characterize the tumor tissue for EGFR and HER3 expression (FIG. 1c).

In this example, the treatment plan for treating HNSCC is to show the effect of SI-B001 in combination with Osimertinib in a dose escalation in HCC827_936 derived xenograft model. The result shows that Osimertinib can achieve some level of cancer control, but mice treated with this TKI ultimately relapsed. Whereas, when combined with 2 of 3 subtherapeutic doses of SI-B001 tested, durable control can be achieved. When Osimertinib was combined with a dose of SI-B001 that achieves stable disease, durable control can be achieved. By Day 31, a significant difference (p<0.05) in tumor volume was evident between all three combinations including SI-B001 when combined with the example TKI Osimertinib (Table 3).

These results demonstrated that the combination of SI-B001 can achieve significantly advantageous therapeutic capability when combined with TKIs, which provides the support for clinical trials in humans (Table 1).

### Example 3. SI-B001 and Osimertinib Combination Therapy in NCI-H1975 Model

To evaluate the anti-tumor efficacy and safety of the experimental drugs SI-B001 in combination with Osimertinib, 80 female nude mice were subcutaneously implanted with 5×10⁶ NCI-H1975 cells and the cells were resuspended in PBS (0.1 ml/mouse). When the tumor grew to an average volume of about 170 mm³, randomly grouped according to tumor size and mouse weight to establish human head and neck squamous cell carcinoma cells NCI-H1975 xenograft model.

Saline and SI-B001 were administered by intravenous infusion once a week. SI-B001 was given at 3 different dose regimens, 9 mg/kg, 16 mg/kg, and 28 mg/kg. DMSO and Osimertinib were administered were given orally once a week. Osimertinib was given at a fixed dose regimen, 96 mg/kg (Table 4).

Tumor volume (mm³) V=½×(a×b)², where a represents the long diameter and b represents the short diameter. Relative tumor volume (RTV), RTV=Vt/V0, where V0 is the tumor volume at time 0 and Vt is the tumor volume after treatment at time t. The T/C value (%) is an indicator of the tumor response to treatment and is the most used evaluation indicator. T/C %=T_{RTV}/C_{RTV}×100%, where T_{RTV} is treatment group average RTV and C_{RTV} is control group average RTV. The T/C value (%) can also be calculated as T/C %=T_{TW}/C_{TW}×100%, where T_{TW} is the average tumor weight at the end of the treatment group experiment and C_{TW} is the average tumor weight at the end of the control group experiment. The relative tumor inhibition rate (Tumor growth inhibition, TGI) is one of the indicators of the tumor response to treatment, TGI %=100%−T/C %.

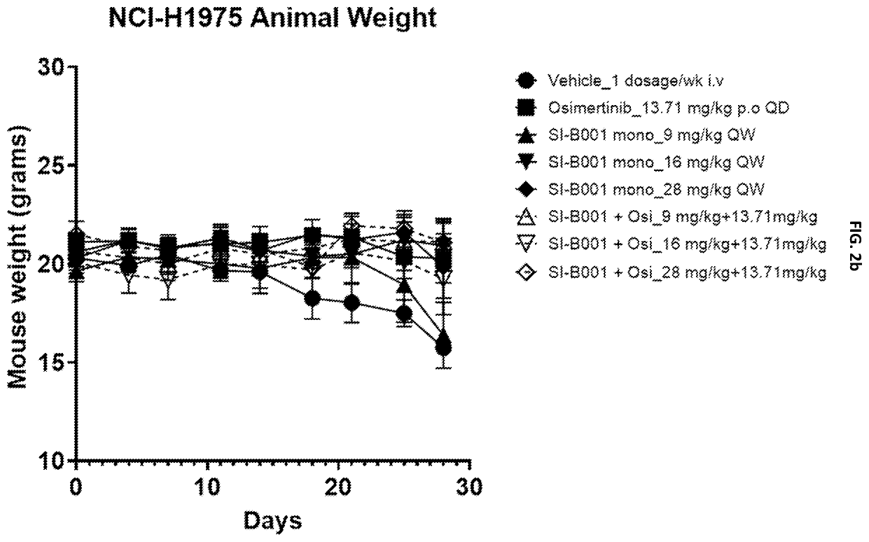

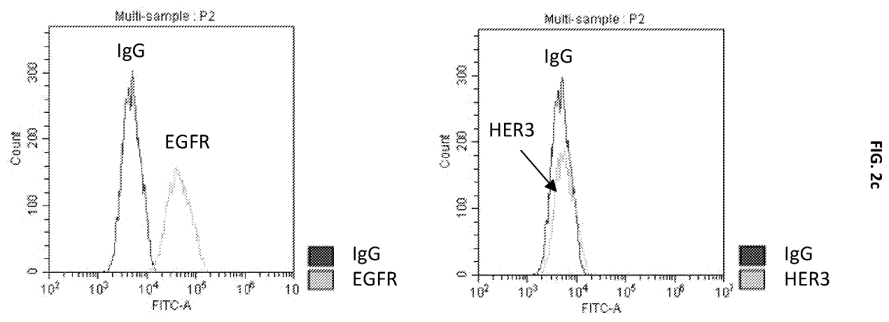

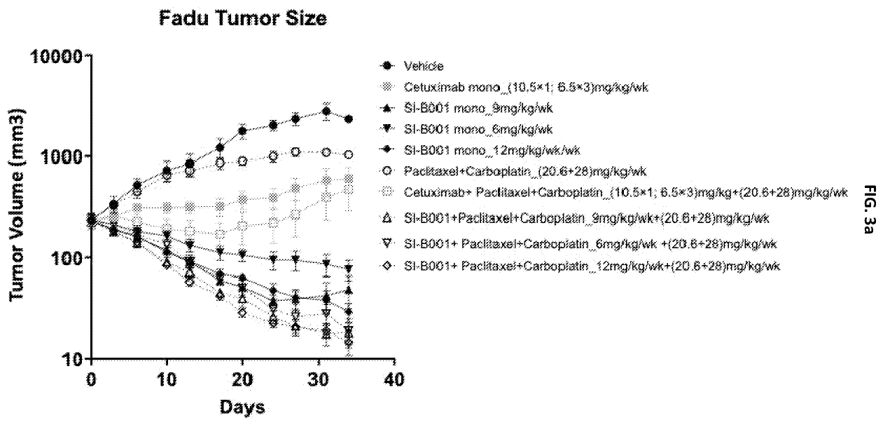

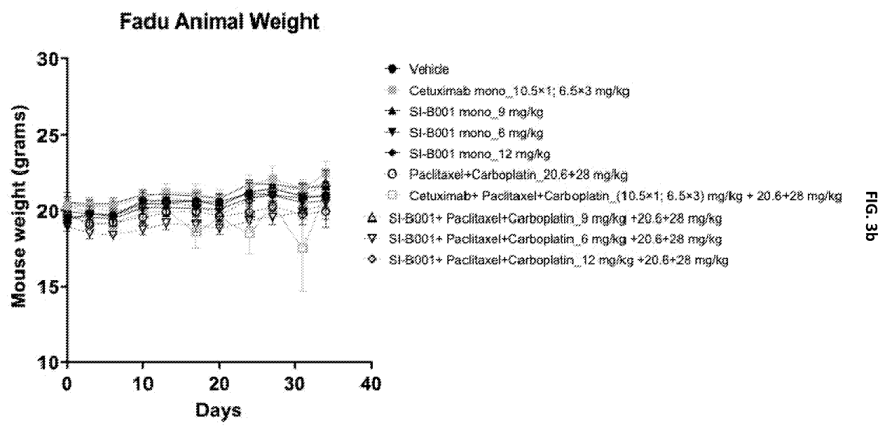

The average tumor volume of mice in the vehicle control group was 2316.19 mm³ on day 25, the average tumor volume of the control Osimertinib single-agent group was 75.02 mm³ on day 25, and TGI (%) was 95.35% (p=0.030). The average tumor volume on the 25^{th} day of SI-B001 single-agent, dose group 9/16/28 mg/kg/mouse, was 253.83 mm3, 173.94 mm3, and 186.08 mm³ respectively, and TGI (%) was 81.79%, 86.85% and 84.80% respectively (p=0.027, p=0.021, p=0.021). The combination of SI-B001 and Osimertinib in each dose group (9/16/28+96 mg/kg/mouse) corresponds to the average tumor volume on day 25 of 49.36 mm³, 9.75 mm³, 8.68 mm³, TGI % were 97.77%, 99.41%, and 99.54% (compared to the single-agent SI-B001 at the corresponding dose level, p=0.243, p=0.409, p=0.399; compared to the single-agent Osimertinib, p=0.633, p=0.096, p=0.092), the anti-tumor effect is better than the corresponding single-agent SI-B001 and single-agent Osimertinib (FIG. 2a, Table 5). In summary, all dose group of SI-B001 combined with Osimertinib have a significant higher anti-tumor effect. The anti-tumor effect is better than the corresponding single-agent SI-B001 and single-agent Osimertinib, showing a significant synergistic anti-tumor effect. And the level of tumor inhibition is positively associated with increased dose of SI-B001.

The weight of mice was measured across the experiment (FIG. 2b). In the vehicle group #1, 1 mouse died before D25, the mortality rate was 20%. In each SI-B001 single-agent groups #3, #4 and #5, 2 mouse died before D25, the mortality rate was 40%. In the Osimertinib single-agent group #2, no mouse died before D25. In the combination group #6, #7, #9, no mouse, 1 mouse and 1 mouse died before D25. All survived mice in all groups showed no significant difference in weight loss during the experiment. There is no clear trend among the groups in terms of number of deaths. This showed no obvious toxicity when combining SI-B001 and Osimertinib.

To verify tumor tissue targets in tumor-bearing mice at the end of the experiment, the tumor tissues were taken after the mice were euthanized, and FACS analysis was used to characterize the EGFR and HER3 expressions in tumor tissue (FIG. 2c).

In this example, SI-B001 (EGFR-HER3 bi-specific antibody) combination with Osimertinib demonstrates increased drug activity when administered in combination for the treatment of the Lung cancer cell line NCI-H1975, as an implanted tumor xenograft in Balb/c-nu mice. In the model, Osimertinib and SI-B001 treatments as single agents maintain relatively stable disease as measured by tumor volume. Whereas, when Osimertinib is combined with any of 3 therapeutic doses of SI-B001 tested, significantly enhanced tumor control can be achieved by the last evaluable timepoint (p<0.05) (Table 5). This evidence helps support the clinical trials that the therapeutic capability of SI-B001 treatment in patients may be improved when combined with Osimertinib (Table 1).

### Example 4. SI-B001 and CarboTaxol (Paclitaxel and Carboplatin) Combination Therapy in Fadu Model

To evaluate the anti-tumor efficacy and safety of the experimental drugs SI-B001 in combination with Paclitaxel and Carboplatin, 80 female nude mice were subcutaneously implanted with 5×10⁶ FaDu cells and the cells were resuspended in PBS (0.1 ml/mouse). When the tumor grew to an average volume of about 230 mm3, randomly grouped according to tumor size and mouse weight to establish human head and neck squamous cell carcinoma cells FaDu xenograft model.

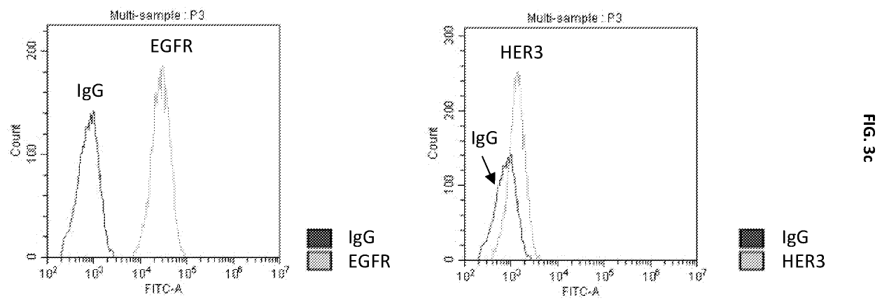

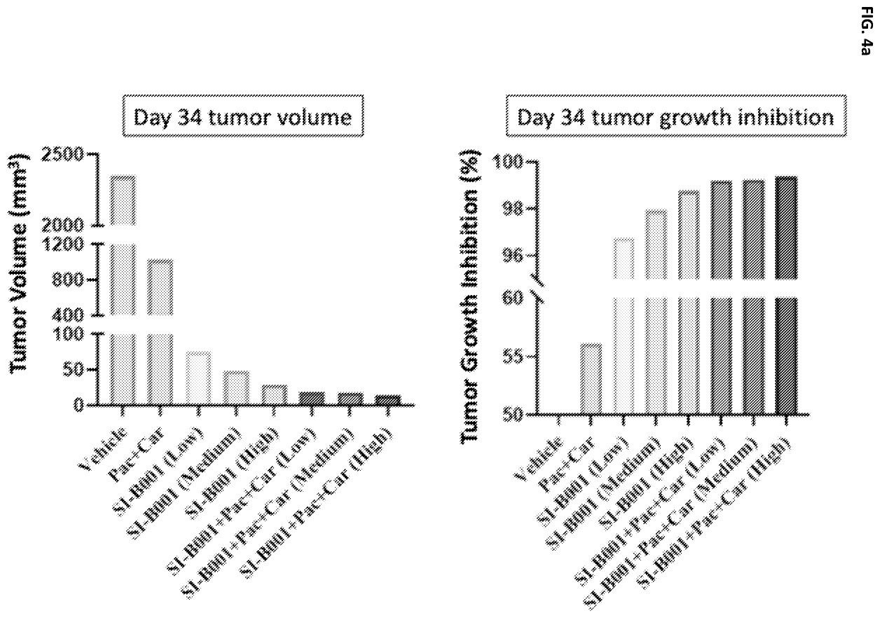

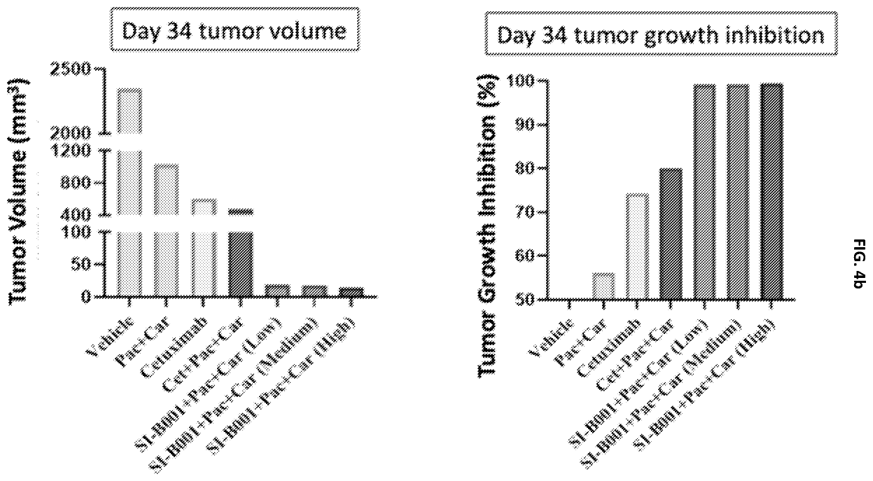

Saline (vehicle control), Cetuximab, SI-B001, paclitaxel and carboplatin were administered by intravenous infusion once a week. SI-B001 was given at 3 different dose regimens, 6 mg/kg, 9 mg/kg, and 12 mg/kg. Cetuximab was given 10.5 mg/kg on day 0 followed by subsequent three doses of 6.5 mg/kg weekly. Paclitaxel and carboplatin were given at a fixed dose regimen, 20.6 mg/kg and 28 mg/kg respectively (FIG. 3a, Table 6).

Tumor volume (mm3) V=½×(a×b)², where a represents the long diameter and b represents the short diameter. Relative tumor volume (RTV), RTV=Vt/V0, where V0 is the tumor volume at time 0 and Vt is the tumor volume after treatment at time t. The T/C value (%) is an indicator of the tumor response to treatment and is the most used evaluation indicator. T/C %=T_{RTV}/C_{RTV}×100%, where T_{RTV} is treatment group average RTV and C_{RTV} is control group average RTV. The T/C value (%) can also be calculated as T/C %=T_{TW}/C_{TW}×100%, where T_{TW} is the average tumor weight at the end of the treatment group experiment and C_{TW} is the average tumor weight at the end of the control group experiment. The relative tumor inhibition rate (Tumor growth inhibition, TGI) is one of the indicators of the tumor response to treatment, TGI %=100%−T/C %.

### SI-B001 vs Chemo vs SI-B001+Chemo

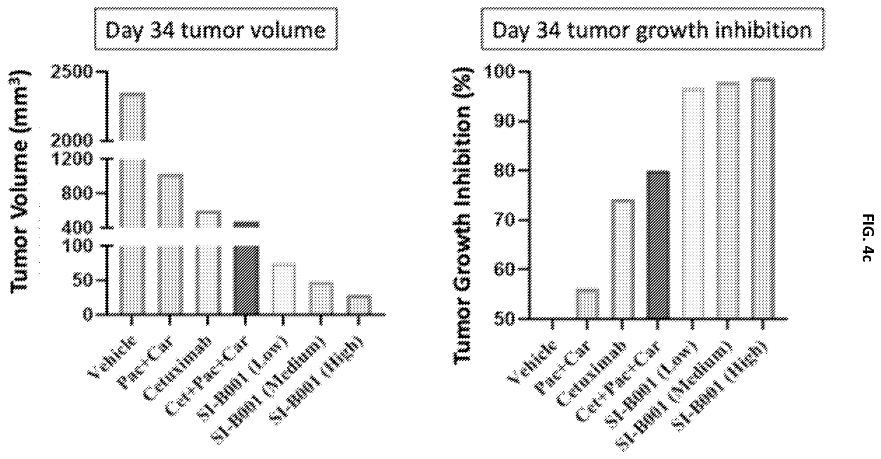

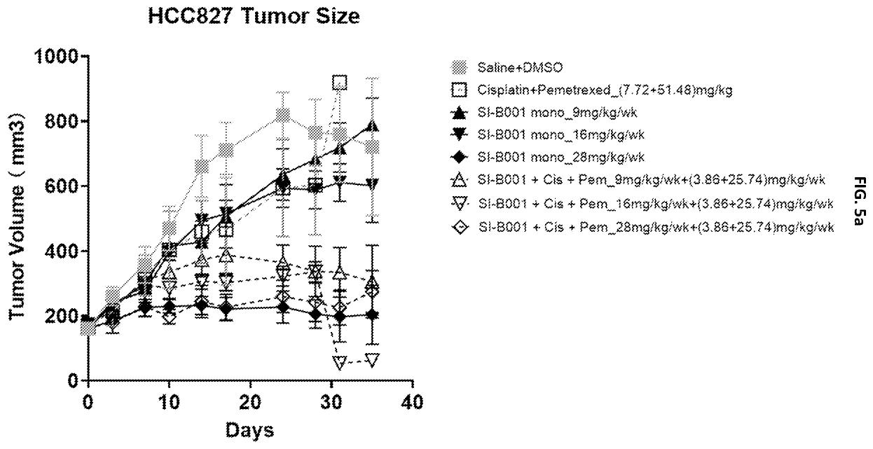

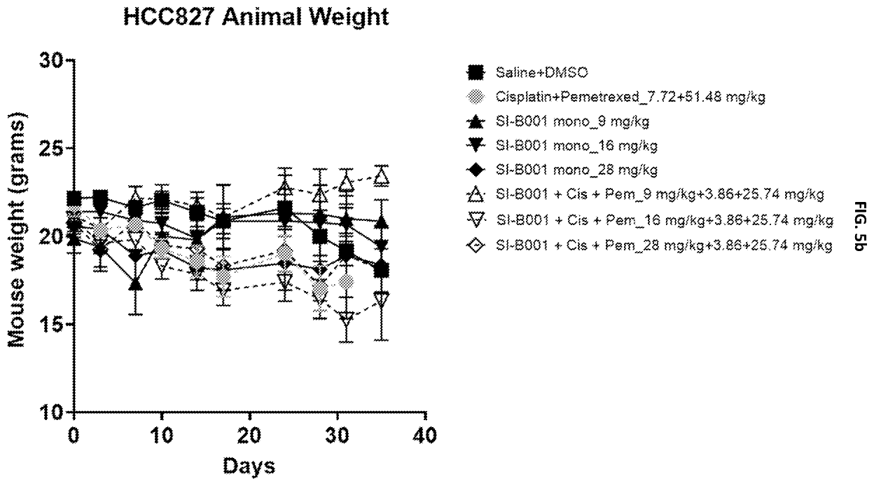

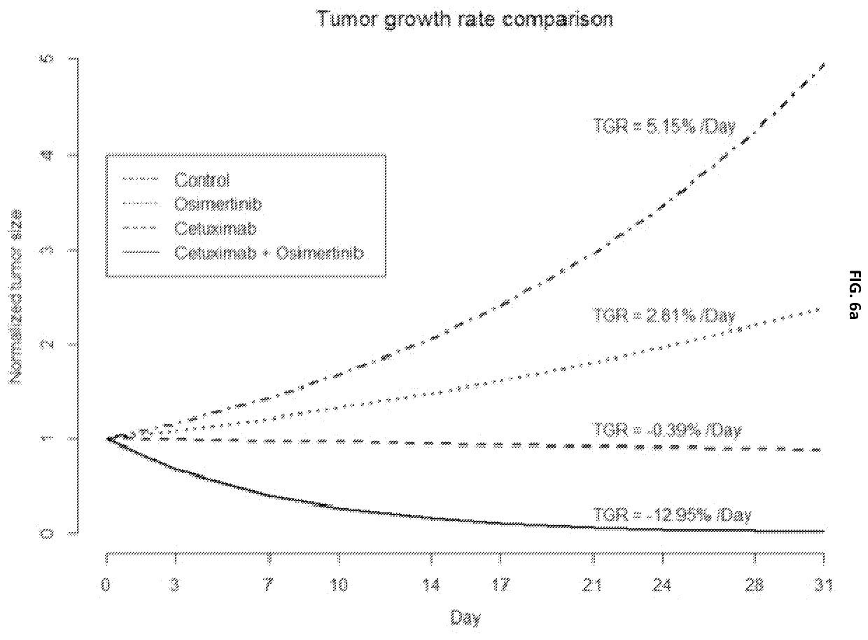

All dose group (6, 9, 12 mg/kg) of SI-B001 single agent have significant anti-tumor effect, the TGI are 96.76%, 97.9% and 98.8%, respectively. The control chemotherapy paclitaxel+carboplatin has a good anti-tumor effect with TGI of 56.16%; All dose group of SI-B001+paclitaxel+carboplatin (6, 9, 12+20.6+28 mg/kg) are significantly more efficacious than control chemotherapy group and SI-B001 single-agent groups with TGI of 99.2%, 99.23% and 99.38% respectively. It shows that the combination of SI-B001+paclitaxel+carboplatin has a synergistic anti-tumor effect in the human head and neck squamous cell FaDu xenograft model (FIG. 4a).

### SI-B001+Chemo vs Cetuximab+Chemo

All dose group (6, 9, 12 mg/kg) of SI-B001 single agent have significant anti-tumor effect, the TGI are 96.76%, 97.9% and 98.8%, respectively. The control cetuximab single-agent with TGI of 74.27% is significantly weaker than the SI-B001 single-agent groups. The control combination cetuximab+paclitaxel+carboplatin has a good anti-tumor effect with a TGI of 79.98% but is significantly weaker than all SI-B001 single-agent groups (96.76%, 97.9% and 98.8%) and SI-B001 chemotherapy combination groups (99.2%, 99.23% and 99.38%) (FIG. 4b, 4c).

The weight of mice was measured across the experiment. There was no death of mice in all SI-B001 single-agent group, and no significant weight loss found (FIG. 3b). It shows that SI-B001 single-agent has low toxicity and good safety. The chemotherapy paclitaxel+carboplatin caused death of 4 mice before the end of the experiment, with a mortality rate of 80%, and 3 of them were found to have a severe weight loss (>20%) before death. The body weight of mice in the chemotherapy paclitaxel+carboplatin group fluctuated significantly. It shows that the chemotherapy paclitaxel+carboplatin is more toxic and is less safe than SI-B001 single-agent. There was no death of mice in SI-B001 and chemotherapy combination, Cetuximab and chemotherapy combination, and no significant weight loss found. It shows that both SI-B001+chemotherapy and Cetuximab+chemotherapy are well tolerated and with good safety.

To verify tumor tissue targets in tumor-bearing mice at the end of the experiment, the tumor tissues were taken after the mice were euthanized, and FACS analysis was used to characterize the EGFR and HER3 expressions in tumor tissue (FIG. 3c).

In summary, SI-B001 (EGFR-HER3 bi-specific antibody) combination with CarboTaxol (Paclitaxel and Carboplatin) demonstrates increased drug activity in combination in the treatment of the head and neck squamous FaDu cell line as an implanted tumor xenograft in Balb/c-nu mice. In this model, CarboTaxol (Paclitaxel and Carboplatin) can delay tumor growth rate but is unable to achieve any measure of tumor regression. Whereas when combined with any of 3 therapeutic doses of SI-B001 tested, enhanced control can be achieved (FIG. 3a). On day 17, when all animals are evaluable for tumor volume, a significant difference (p<0.05) in tumor volume is evident between all three combinations of CarboTaxol (Paclitaxel and Carboplatin) and SI-B001 (Table 7). Based on this evidence the combination of SI-B001 can achieve increased therapeutic capability when combined with chemotherapy combinations.

### Example 5. SI-B001 and Cis/Pem Combination Therapy in HCC827 Model

To evaluate the anti-tumor efficacy and safety of the experimental drugs SI-B001 in combination with Cis/Pem, 40 female nude mice were subcutaneously implanted with 5×10⁶ HCC827_936 cells and the cells were resuspended in PBS (0.1 ml/mouse). When the tumor grew to an average volume of about 163 mm3, randomly grouped according to tumor size and mouse weight to establish human head and neck squamous cell carcinoma cells HCC827_936 xenograft model.

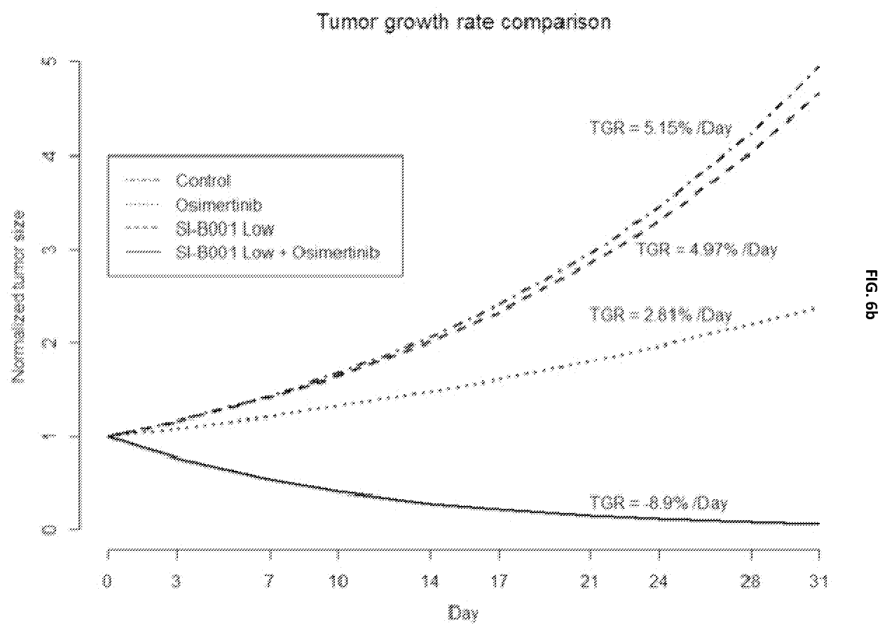

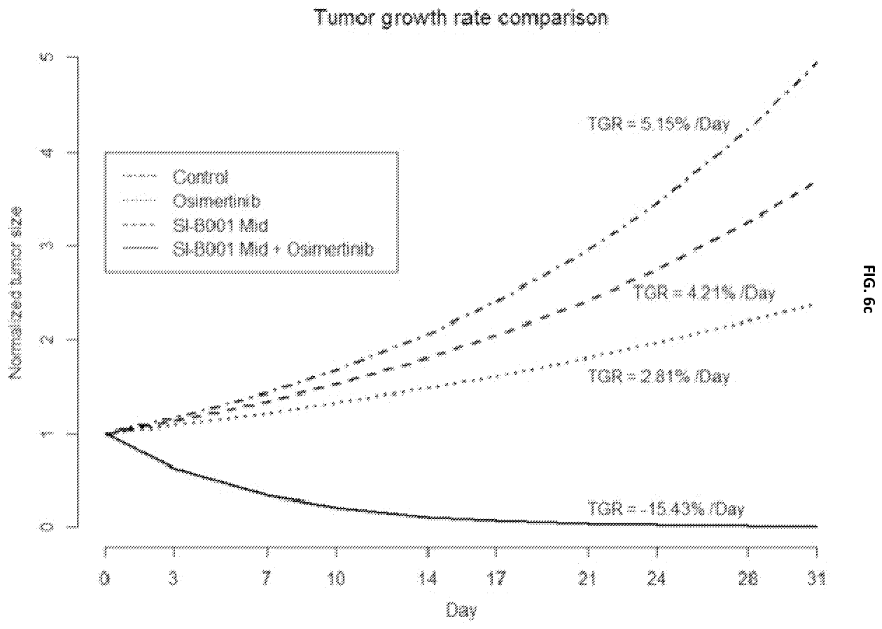

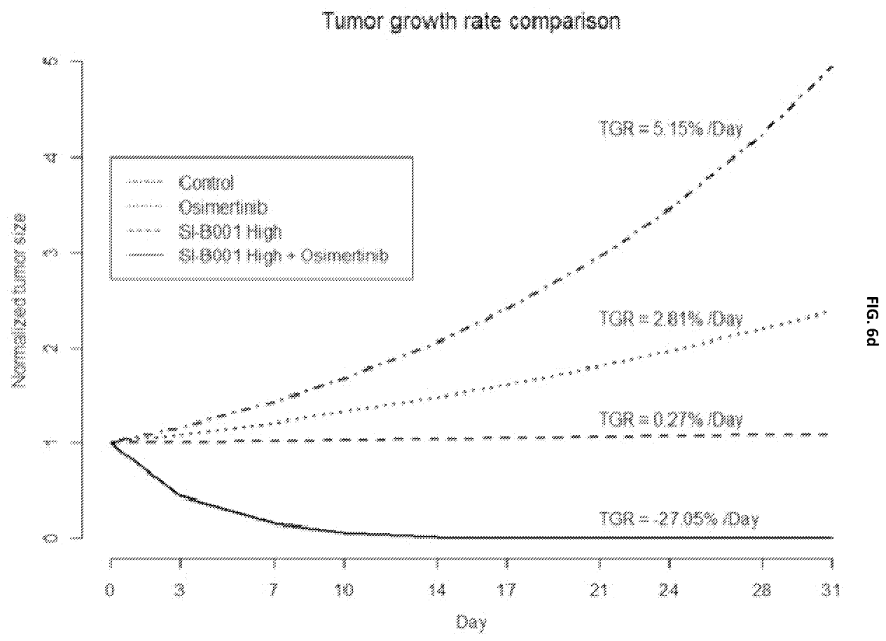

Saline (vehicle control), SI-B001, cisplatin and pemetrexed were administered by intravenous infusion once a week. SI-B001 was given at 3 different dose regimens, 9 mg/kg, 16 mg/kg, and 28 mg/kg. Cisplatin and pemetrexed were given at 7.72 mg/kg and 51.48 mg/kg when combined with cetuximab or SI-B001, and 3.86 mg/kg and 25.74 mg/kg alone (FIG. 5a; Table 8).

Tumor volume (mm³) V=½×(a×b)², where a represents the long diameter and b represents the short diameter. Relative tumor volume (RTV), RTV=Vt/V0, where V0 is the tumor volume at time 0 and Vt is the tumor volume after treatment at time t. The T/C value (%) is an indicator of the tumor response to treatment and is the most used evaluation indicator. T/C %=T_{RTV}/C_{RTV}×100%, where T_{RTV} is treatment group average RTV and C_{RTV} is control group average RTV. The T/C value (%) can also be calculated as T/C %=T_{TW}/C_{TW}×100%, where T_{TW} is the average tumor weight at the end of the treatment group experiment and C_{TW} is the average tumor weight at the end of the control group experiment. The relative tumor inhibition rate (Tumor growth inhibition, TGI) is one of the indicators of the tumor response to treatment, TGI %=100%−T/C %.

The average tumor volume of mice in the vehicle control group was 765.1 mm³ on day 24, the average tumor volume of the control Cis/Pem group was 603.1 mm³ on day 24, and TGI (%) was 21.2% (p=0.473). The average tumor volume on day 24 of SI-B001 single-agent, dose group 9/16/28 mg/kg/mouse, was 682. mm³, 588.1 mm³, and 205.7 mm³ respectively, and TGI (%) was 10.8%, 23.1% and 73.1% respectively (compared to the vehicle control group, p=0.562, p=0.198, p=0.007). The combination of SI-B001 and Cis/Pem in each dose group (9/16/28+3.86+25.74 mg/kg/wk) corresponds to the average tumor volume on day 24 of 337.7 mm³, 335.6 mm³, 241.8 mm³, TGI % were 55.9%, 56.1%, and 68.4% (compared to the single-agent SI-B001 at the corresponding dose level, p=0.032, p=0.030, p=0.652; compared to Cis/Pem, p=0.297, p=0.321, p=0.222), the anti-tumor effect is better than the corresponding single-agent SI-B001 except for the high dose of SI-B001, and is better than Cis/Pem alone.

In summary, SI-B001 combined with Cis/Pem have an overall higher anti-tumor effect (FIG. 5a, Table 9). The anti-tumor effect is better than single-agent SI-B001 and Cis/Pem alone, showing a synergistic anti-tumor effect. And the level of tumor inhibition is positively associated with increased dose of SI-B001.

To assess the safety issue, the weight of mice was measured across the experiment (FIG. 5b). In the vehicle group #1, 1 mouse died before D24, the mortality rate was 20%. In SI-B001 single-agent low dose groups #3, 1 mouse died before D24, the mortality rate was 20%. In SI-B001 single-agent middle dose groups #4, 2 mouse died before D24, the mortality rate was 40%. In SI-B001 single-agent high dose groups #5, 2 mice died before D24, the mortality rate was 40%. In the Cis/Pem group #2, 3 mice died before D24, the mortality rate was 60%. In the combination group #6 and #8, SI-B001 low dose and high dose, 2 mice died before D24, the mortality rate was 40%. In the combination group #7, SI-B001 mid dose, 1 mouse died before D24, the mortality rate was 20%. There is no clear trend among the groups in terms of number of deaths. This showed no obvious toxicity when combining SI-B001 and Cis/Pem.

SI-B001 in combination with Cis/Pem demonstrates increased drug activity when administered in combination for the treatment of the lung cancer derived HCC827 cell line as an implanted tumor xenograft in Balb/c-nu mice. In this model, Cis/Pem can delay tumor growth rate at the early evaluable timepoints, but the tumor growth is ultimately uncontrolled. As a single agent, SI-B001 shows dose dependent control of tumor growth. When Cis/Pem is combined with subtherapeutic doses of SI-B001, enhanced control can be achieved.

Based on this evidence the combination of SI-B001 can achieve increased therapeutic capability when combined with chemotherapy combinations.

### Example 6: Synergistic Effect of SI-B001 with Either TKI or Chemotherapeutics in Combination Therapy

Following Bliss definition, the combination therapy has synergistic effect if the Bliss independence score is greater than zero. The Bliss definition of synergy was applied to tumor volume data measured over 31 days in the HCC827 xenograft Balb/c-nu mouse model. In this model, mice were treated with Cetuximab, SI-B001 Low (9 mg/kg), SI-B001 Mid (16 mg/kg), SI-B001 High (28 mg/kg), platinum-based double chemotherapy (cisplatin and pemetrexed), the 3rd generation TKI (Osimertinib) and their combinations. Cetuximab, chemotherapy and Osimertinib were given at the clinical equivalent dose (Table 9).

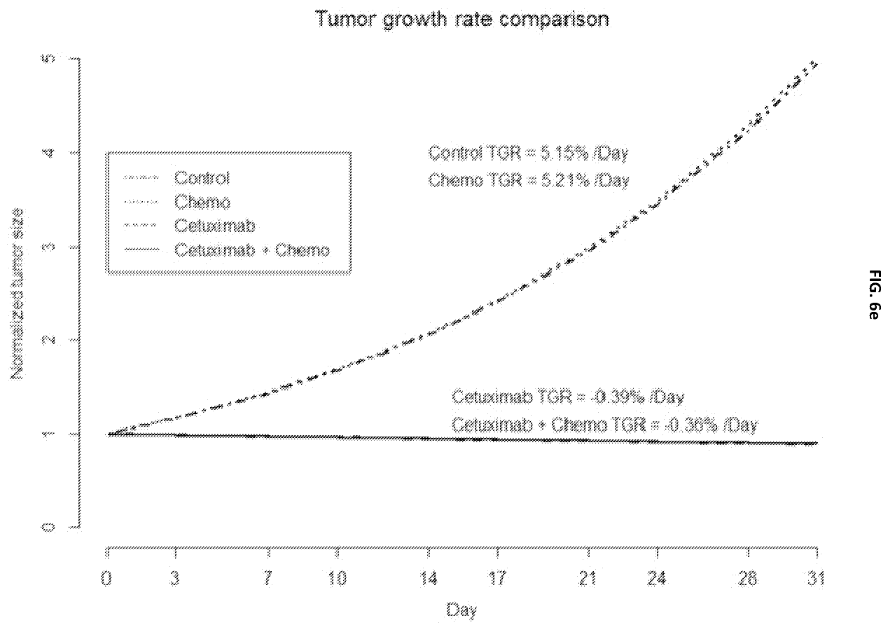

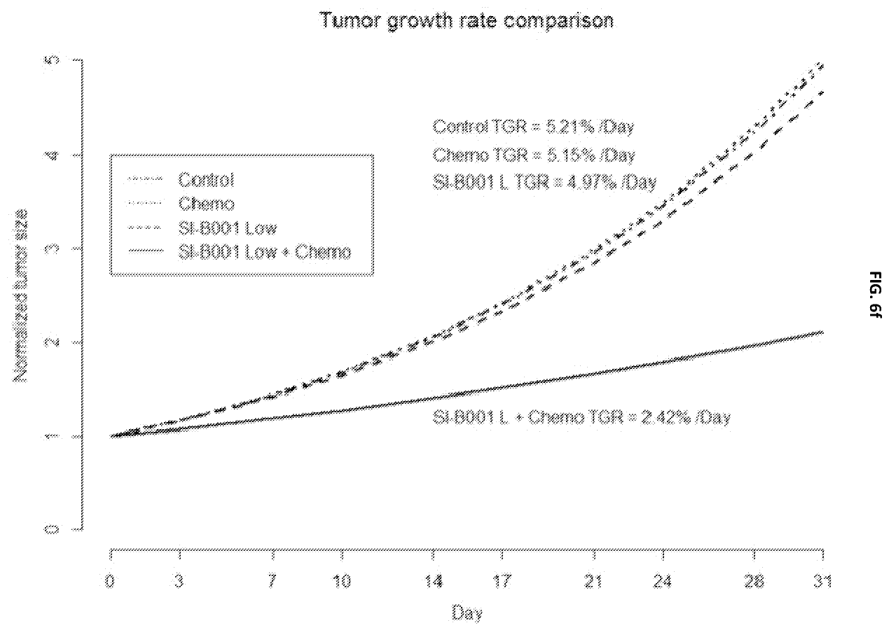

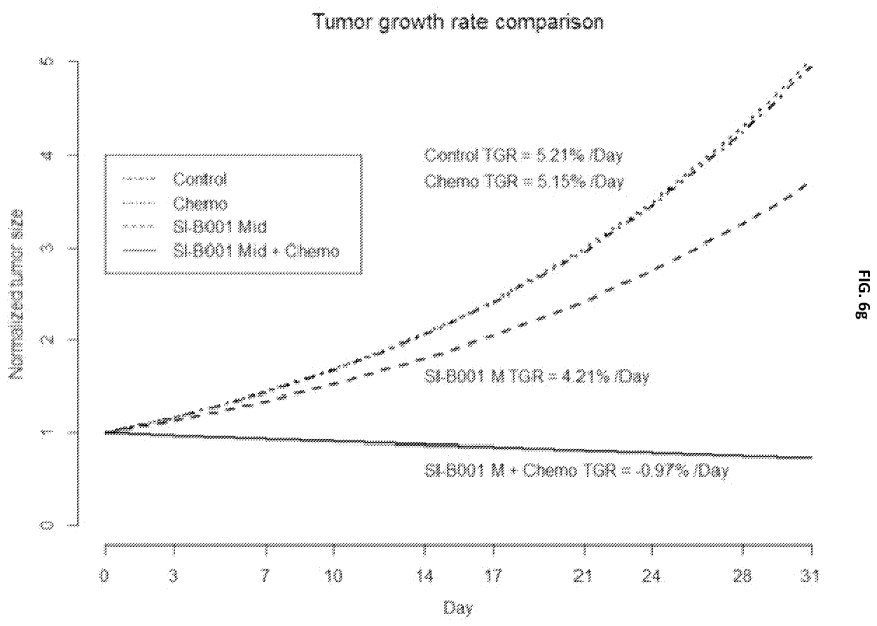

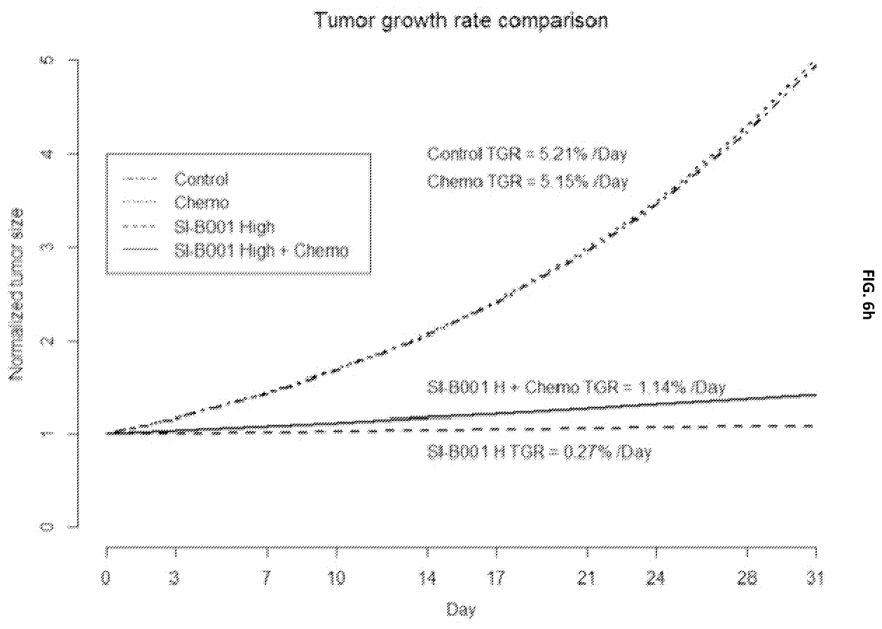

The tumor growth rate in each treatment group was calculated using linear mixed model and the same assumptions as specified in Demidenko, 2019. The tumor growth comparisons of the combinations versus individual agents are demonstrated in FIG. 6a-6h. When Cetuximab and SI-B001 were combined with Osimertinib, all showed significant synergistic effect (Table 10). The synergy of SI-B001 with Osimertinib increases as the dose of SI-B001 increases and is stronger than the synergistic effect of Cetuximab with Osimertinib. The combination of SI-B001 high dose with Osimertinib exhibits the best overall efficacy. When Cetuximab and SI-B001 were combined with the chemotherapy, all showed significant synergistic effect except for SI-B001 high dose (Table 11). The combination of SI-B001 mid dose with the chemotherapy exhibits the best overall efficacy.

These results demonstrate that the combination of SI-B001 can achieve increased therapeutic capability when combined with TKIs, which provides the support for clinical trials in humans (Table 1).

### Example 7: Clinical Studies of SI-B001 Combined with Chemotherapeutics as the Treatment in Solid Tumors

SI-B001 combined with chemotherapies is being tested in NSCLC, HNSCC, and ESCC in clinical studies (Table 1, S201, S206, and S207, respectively).

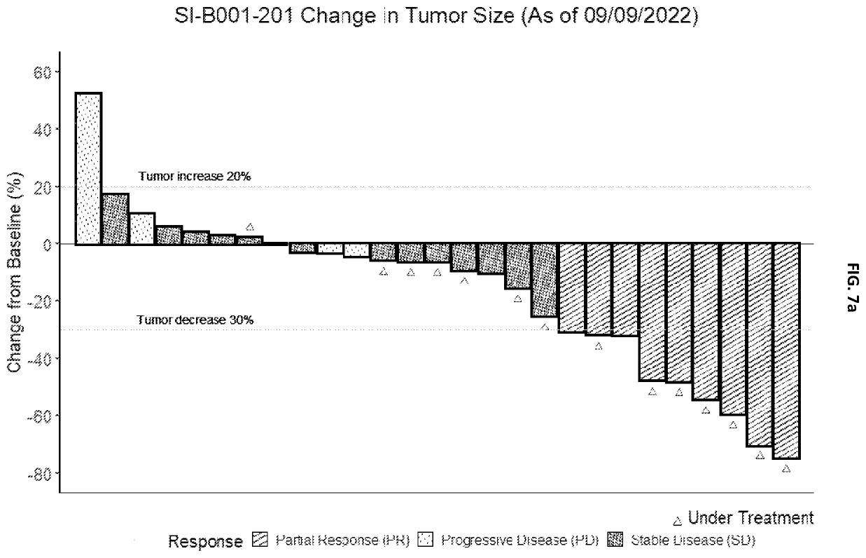

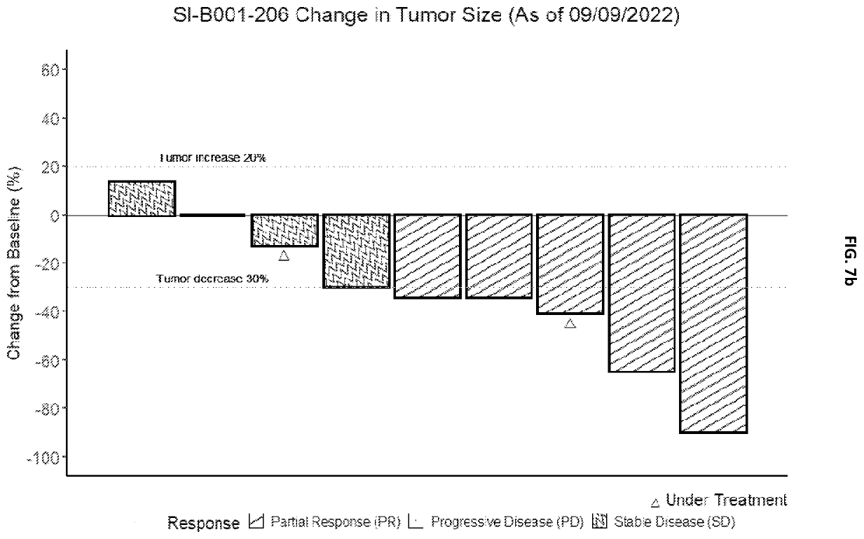

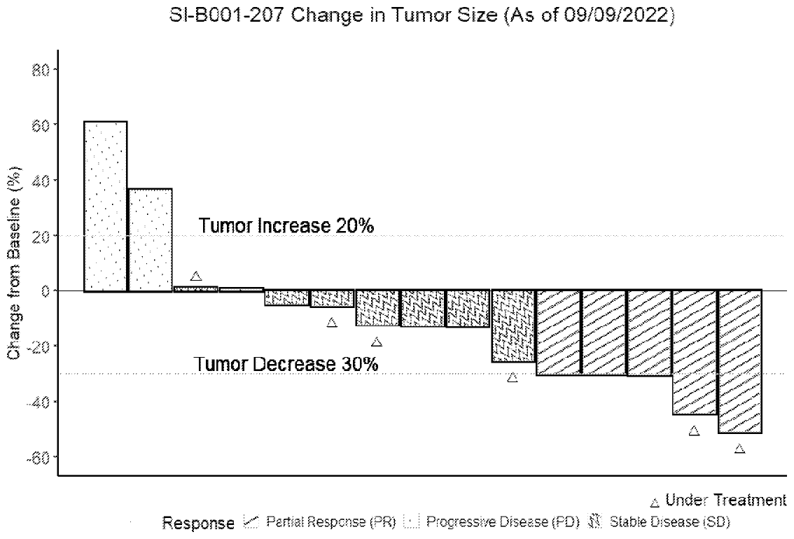

In the S201 study, SI-B001 was combined with AP (Cis/Pem, cisplatin+pemetrexed) or TP (CarboTaxol, carboplatin+paclitaxel) in 2nd line NSCLC patients who have only received anti-PD-1/L1 monotherapy in the first line treatment, and SI-B001 was combined with docetaxel in 2nd/3rd lines NSCLC patients who have been exposed to both platinum-based chemotherapy and anti-PD-1/L1 therapy. A preliminary report revealed that of 46 patients have been enrolled, 91% were male, the median age was 62.5 years (range, 33-76 years). Among the 46 patients, 1 was treated with SI-B001+AP/TP, 45 were treated with SI-B001+docetaxel, the median number of previous lines of therapy was 2. Among the 46 patients, 27 were eligible for efficacy assessment (with at least 1 post baseline tumor assessment) among whom 14 were still under treatment. The overall response rate (ORR) was 33.3% (95% Cl, 16.5 to 54.0) and the disease control rate (DCR) was 81.5% (95% Cl, 61.9 to 93.7). FIG. 7a shows the tumor responses in the waterfall plot. The efficacy of SI-B001 combined with chemotherapy in this indication compared to chemotherapy alone (Schuette et al., 2005) indicated the potential benefit in this patient population.

In the S206 study, SI-B001 is combined with paclitaxel in 2nd/3rd line HNSCC patients who were resistant to front line(s) chemotherapy and anti-PD-1/L1 therapy. A preliminary report revealed that of 23 patients have been enrolled, 87% were male, the median age was 56 years (range, 36-75 years). All patients have received one prior line treatment with chemotherapy plus immunotherapy. Among the 23 patients, 9 were eligible for efficacy assessment (with at least 1 post baseline tumor assessment) among whom 2 were still under treatment. The overall response rate (ORR) was 55.6% (95% Cl, 21.2 to 86.3) and the disease control rate (DCR) was 88.9% (95% Cl, 51.8 to 99.7). FIG. 7b shows the tumor responses in the waterfall plot. Compared with the historical data from cetuximab plus chemotherapy (Issa et al., 2021), a better response rate by SI-B001+paclitaxel treatment seems to be a trend leading to more clinical benefits in this patient population.

In the S207 study, SI-B001 is combined with irinotecan in 2nd line ESCC patients who were resistant to front line platinum-based chemotherapy and anti-PD-1/L1 therapy. A preliminary report revealed that of 21 patients have been enrolled, 90% were male, their median age was 58 years (range, 48-70 years). Among the 21 patients, 15 were eligible for efficacy assessment (with at least 1 post baseline tumor assessment) among whom 6 were still under treatment. The overall response rate (ORR) was 33.3% (95% Cl, 11.8 to 61.6) and the disease control rate (DCR) was 80.0% (95% Cl, 51.9 to 95.7). The tumor responses are showed in the waterfall plot (FIG. 7c). Based on the preliminary data, the efficacy of SI-B001 plus irinotecan may be better than the retrospective data of irinotecan alone or combined with other chemotherapies in pretreated ESCC patients (Burkart et al., 2007).

In summary, SI-B001 combined with chemotherapies were well-tolerated across all three studies. No treatment related death has been observed. Thus, the efficacy and safety data support further development of SI-B001 in these indications.

### REFERENCES

Robichaux J P, Le X, Vijayan R S K, et al. Structure-based classification predicts drug response in EGFR-mutant NSCLC. Nature. 2021 September; 597(7878):732-737.
Wu L, Ke L, Zhang Z, Yu J, Meng X. Development of EGFR TKis and Options to Manage Resistance of Third-Generation EGFR TKI Osimertinib: Conventional Ways and Immune Checkpoint Inhibitors. Front Oncol. 2020 Dec. 18; 10:602762.
Rebuzzi S E, Alfieri R, La Monica S, Minari R, Petronini P G, Tiseo M. Combination of EGFR-TKIs and chemotherapy in advanced EGFR mutated NSCLC: Review of the literature and future perspectives. Crit Rev Oncol Hematol. 2020 February; 146:102820.
Demidenko, E., & Miller, T. W. (2019). Statistical determination of synergy based on Bliss definition of drugs independence. PLoS One, 14(11), e0224137.
Chong C R, Janne P A. The quest to overcome resistance to EGFR-targeted therapies in cancer. Nat Med 2013; 19:1389-400.
Lim S M, Syn N L, Cho B C, et al. Acquired resistance to EGFR targeted therapy in non-small cell lung cancer: Mechanisms and therapeutic strategies. Cancer Treat Rev 2018; 65:1-10.
Sullivan, I and Planchard, D. (2017) Next-generation EGFR tyrosine kinase inhibitors for treating EGFR-mutant lung cancer beyond first line. Front. Med. 3:76.
Schuette, W., Nagel, S., Blankenburg, T., et al. (2005). Phase Ill study of second-line chemotherapy for advanced non-small-cell lung cancer with weekly compared with 3-weekly docetaxel. Journal of Clinical Oncology, 23(33), 8389-8395.
Issa, M., Klamer, B., Karivedu, V., et al. (2021). Use of cetuximab added to weekly chemotherapy to improve progression-free survival in patients with recurrent metastatic head and neck squamous cell carcinoma after progression on immune checkpoint inhibitors. Journal of Clinical Oncology>List of Issues>Volume 39, Issue 15_suppl.
Burkart, C., Bokemeyer, C., Klump, B., et al. (2007). A phase II trial of weekly irinotecan in cisplatin-refractory esophageal cancer. Anticancer research, 27(4C), 2845-2848.

### Tables

TABLE 1
Exemplary therapies for human cancer, including but not limited
to, solid tumors, NSCLC, HNSCC, and ESCC.

| Cancer | Therapeutic Agent | Dosing regimen |
|---|---|---|
| Solid | 1) SI-B001 monotherapy; | SI-B001 is administered by |
| tumors | 2) SI-B001 plus | intravenous drip at least |
| | Osimertinib | once weekly (QW), |
| | | bi-weekly or every other |
| | | week(Q2W) |
| | | Every three weeks (Q3W), |
| | | or Day 1 and Day 8 every |
| | | three weeks (D1D8Q3W) |
| | | in a dosage of, for example, |
| | | 6 mg/kg, 9 mg/kg, 12 mg/kg, |
| | | 14 mg/kg, 16 mg/kg, or |
| | | 21 mg/kg; 120 min ± 10 |
| | | min after the first |
| | | intravenous drip, if the |
| | | infusion reaction is tolerable |
| | | during the first dose, the |
| | | subsequent infusion can be |
| | | completed within 60-120 |
| | | min; and if SI-B001 and |
| | | chemotherapy/target therapy |
| | | are used on the same |
| | | day, the infusion of drugs |
| | | should be continued after |
| | | the completion of SI-B001 |
| | | infusion. |
| NSCLC²⁰¹ | 1) SI-B001 plus AP | The dose of SI-B001 is |
| | (pemetrexed plus | administered by intravenous |
| | cisplatin) | drip at least once |
| | 2) SI-B001 plus TP | weekly (QW); and the |
| | (paclitaxel plus cisplatin) | administration of AP, TP, |
| | 3) SI-B001 plus | or Docetaxel should refer |
| | Docetaxel | to the drug instructions |
| | | and standard usage and |
| | | should be administered |
| | | immediately after SI-B001 is |
| | | completed. |
| HNSCC²⁰⁶ | 1) SI-B001 | The dose of SI-B001 is |
| | plus Paclitaxel | administered by intravenous |
| | | drip once a week (QW); |
| | | the dosage of paclitaxel is |
| | | 80 mg/m2 QW. SI-B001 |
| | | and paclitaxel are used on |
| | | the same day. After SI-B001 |
| | | infusion, paclitaxel is |
| | | pretreated and injected for |
| | | no less than 3 hours. |
| ESCC²⁰⁷ | 1) SI-B001 plus | The dose of SI-B001 is |
| | irinotecan | administered by intravenous |
| | | drip every 2 weeks (Q2W); |
| | | and the dose of |
| | | irinotecan is 180 mg/m2 |
| | | Q2W, the infusion method is |
| | | according to the drug |
| | | instructions, SI-B001 and |
| | | irinotecan are used on the |
| | | same day, and irinotecan |
| | | is injected after SI-B001 infusion. |

Exemplary indications of human cancer:
²⁰¹Locally advanced or metastatic EGFR wild-type ALK wild-type NSCLC patients with disease progression or intolerance to first-line therapy containing anti-PD-1/L1 antibody or post-line therapy containing anti-PD-1/L1;
²⁰⁶Recurrent and metastatic HNSCC (non-nasopharyngeal) patients progressed on or intolerant to prior anti-PD-1/L1 monoclonal antibody with/without platinum-based chemotherapy (previously received at most 2 lines of systemic therapy); and
²⁰⁷Recurrent and metastatic ESCC patients with disease progression on or intolerance to anti-PD-1/L1 monoclonal antibody with/without chemotherapy.

TABLE 2
Experiment design (Example 2)

| | # of | | Dose | Adminis- | Fre- |
|---|---|---|---|---|---|
| Group | Mouse | Treatment | (mg/kg/wk) | tration | quency |
| 1 | 5 | Vehicle | 0 | i.v. + p.o. | QW/QD |
| | | (saline + DMSO) | | | |
| 2 | 5 | SI-B001 | 9 | i.v. | QW |
| 3 | 5 | SI-B001 | 16 | i.v. | QW |
| 4 | 5 | SI-B001 | 28 | i.v. | QW |
| 5 | 5 | Osimertinib | 96 | p.o. | QD |
| 6 | 5 | SI-B001 + | 9 + 96 | i.v. + p.o. | QW/QD |
| | | Osimertinib | | | |
| 7 | 5 | SI-B001 + Osimertinib | 16 + 96 | i.v. + p.o. | QW/QD |
| 8 | 5 | SI-B001 + Osimertinib | 28 + 96 | i.v. + p.o. | QW/QD |

Note:
The administration volume was 10 ml/kg per mouse. Tumor volume was assessed twice a week.

TABLE 3
Experiment result on Day 31 (Example 2)

| | | | | P-value (Day 31) | | |
|---|---|---|---|---|---|---|
| | Tumor | | | to | to | to |
| | volume | TGI | T/C | group | group | group |
| Group | x ± SD (mm³) | % | % | 1 | 2 | 3 |
| 1 | 720.7 ± 298.7 | N/A | N/A | N/A | N/A | N/A |
| 2 | 789.7 ± 140.7 | −10.7% | 110.7% | 0.783 | 0.009 | N/A |
| 3 | 601.4 ± 112.9 | 24.6% | 75.4% | 0.654 | 0.252 | N/A |
| 4 | 203.9 ± 91.3 | 73.2% | 26.8% | 0.123 | 0.355 | N/A |
| 5 | 344.5 ± 23.6 | 54.9% | 45.1% | 0.216 | N/A | N/A |
| 6 | 46.8 ± 34.3 | 93.7% | 6.3% | 0.085 | 0.007 | 0.002 |
| 7 | 7.8 ± 9.0 | 99.1% | 0.9% | 0.078 | 0.007 | 0.012 |
| 8 | 0.00 ± 0.00 | 100% | 0% | 0.076 | 0.044 | 0.268 |

TABLE 4
Experiment design (Example 3):

| | | | Dose | | |
|---|---|---|---|---|---|
| | # of | | (mg/kg/wk + | Adminis- | Fre- |
| Group | Mouse | Treatment | mg/kg/day) | tration | quency |
| 1 | 5 | Vehicle | 0 | i.v. + p.o | QW/QD |
| | | (saline + DMSO) | | | |
| 2 | 5 | Osimertinib | 96 | p.o | QD |
| 3 | 5 | SI-B001 | 9 | i.v | QW |
| 4 | 5 | SI-B001 | 16 | i.v | QW |
| 5 | 5 | SI-B001 | 28 | i.v | QW |
| 6 | 5 | SI-B001 + Osimertinib | 9 + 13.71 | i.v. + p.o | QW/QD |
| 7 | 5 | SI-B001 + Osimertinib | 16 + 13.71 | i.v. + p.o | QW/QD |
| 8 | 5 | SI-B001 + Osimertinib | 28 + 13.71 | i.v. + p.o | QW/QD |

Note:
The administration volume was 10 ml/kg per mouse. Tumor volume was assessed twice a week.

TABLE 5
Experiment Result on Day 25 (Example 3)

| | | | | P-value (Day 25) | | |
|---|---|---|---|---|---|---|
| | Tumor | | | to | to | to |
| | volume | TGI | T/C | group | group | group |
| Group | x ± SD (mm³) | % | % | 1 | 2 | 3 |
| 1 | 2316.2 ± 564.8 | N/A | N/A | N/A | N/A | N/A |
| 2 | 75.0 ± 60.3 | 95.4% | 4.7% | 0.030 | N/A | N/A |
| 3 | 253.8 ± 1179.6 | 81.8% | 18.2% | 0.027 | 0.292 | N/A |
| 4 | 173.9 ± 224.3 | 86.9% | 13.2% | 0.021 | 0.599 | N/A |
| 5 | 186.1 ± 236.2 | 84.8% | 15.2% | 0.021 | 0.576 | N/A |
| 6 | 49.4 ± 83.4 | 97.8% | 2.2% | 0.029 | 0.633 | 0.243 |
| 7 | 9.8 ± 5.6 | 99.4% | 0.6% | 0.029 | 0.096 | 0.409 |
| 8 | 8.7 ± 7.8 | 99.5% | 0.5% | 0.029 | 0.092 | 0.399 |

TABLE 6
Experiment design (Example 4)

| | # of | | Dose | Adminis- | Fre- |
|---|---|---|---|---|---|
| Group | Mouse | Treatment | (mg/kg/wk) | tration | quency |
| 1 | 5 | Vehicle | 0 | i.v. | QW |
| 2 | 5 | Cetuximab | 10.5-6.5 × 3 | i.v. | QW |
| 3 | 5 | SI-B001 | 6 | i.v. | QW |
| 4 | 5 | SI-B001 | 9 | i.v. | QW |
| 5 | 5 | SI-B001 | 12 | i.v. | QW |
| 6 | 5 | Cetuximab + | (10.5-6.5 × 3) + | i.v. | QW |
| | | Pac + Car | 20.6 + 28 | | |
| 7 | 5 | Pac + Car | 20.6 + 28 | i.v. | QW |
| 8 | 5 | SI-B001 + Pac + Car | 6 + (20.6 + 28) | i.v. | QW |
| 9 | 5 | SI-B001 + Pac + Car | 9 + (20.6 + 28) | i.v. | QW |
| 10 | 5 | SI-B001 + Pac + Car | 12 + (20.6 + 28) | i.v. | QW |

Note:
The administration volume was 10 ml/kg per mouse. Cetuximab dose “10.5-6.5 × 3” indicates 10.5 mg/kg on day 0 followed by subsequent three doses of 6.5 mg/kg weekly; Pac means Paclitaxel; and
Car means Carboplatin; Tumor volume was assessed twice a week.

TABLE 7
Tumor volume comparisons using t-test on day 17 (Example 4)

| | SI-B001 mono | SI-B001 mono | SI-B001 mono |
|---|---|---|---|
| | 6 (mg/kg) | 9 (mg/kg) | (12 mg/kg) |
| | SI-B001 plus | SI-B001 plus | SI-B001 plus |
| SI-B001 mono | Paclitaxel + | Paclitaxel + | Paclitaxel + |
| VS. | Carboplatin | Carboplatin | Carboplatin |
| SI-B001 + | (20.6 + 28) | (20.6 + 28) | (20.6 + 28) |
| Chemo | (mg/kg) | (mg/kg) | (mg/kg) |
| P value | 0.0324 | 0.0471 | 0.013 |
| (t-test, Day 17) | | | |

TABLE 8
Experiment design (Example 5)

| | # of | | | Adminis- | Fre- |
|---|---|---|---|---|---|
| Group | Mouse | Treatment | Dose (mg/kg/wk) | tration | quency |
| 1 | 5 | Vehicle | 0 | i.v. | QW |
| 2 | 5 | Cis/Pem | 7.72 + 51.48 | i.v. | QW |
| 3 | 5 | SI-B001 | 9 | i.v. | QW |
| 4 | 5 | SI-B001 | 16 | i.v. | QW |
| 5 | 5 | SI-B001 | 28 | i.v. | QW |
| 6 | 5 | SI-B001 + | 9 + (3.86 + 25.74) | i.v. | QW |
| | | Cis + Pem | | | |
| 7 | 5 | SI-B001 + | 16 + (3.86 + 25.74) | i.v. | QW |
| | | Cis + Pem | | | |
| 8 | 5 | SI-B001 + | 28 + (3.86 + 25.74) | i.v. | QW |
| | | Cis + Pem | | | |

Note:
The administration volume is 10 ml/kg per mouse. Pem means Pemetrexed; Cis means Cisplatin; Tumor volume is assessed twice a week.

TABLE 9
Experiment Result on Day 24 (Example 5)

| | Tumor | | | P-value (Day 24) | | |
|---|---|---|---|---|---|---|
| | volume | TGI | T/C | to | to | to |
| Group | x ± SD (mm³) | % | % | group 1 | group 2 | group 3 |
| 1 | 765.1 ± 203.5 | N/A | N/A | N/A | N/A | N/A |
| 2 | 603.1 ± 216.3 | 21.2% | 78.8% | 0.473 | N/A | N/A |
| 3 | 682.7 ± 174.7 | 10.8% | 89.2% | 0.562 | 0.702 | N/A |
| 4 | 588.1 ± 101.7 | 23.1% | 76.9% | 0.198 | 0.939 | N/A |
| 5 | 205.7 ± 76.1 | 73.1% | 26.9% | 0.007 | 0.213 | N/A |
| 6 | 337.7 ± 134.0 | 55.9% | 44.1% | 0.021 | 0.297 | 0.032 |
| 7 | 335.6 ± 62.9 | 56.1% | 43.9% | 0.020 | 0.321 | 0.030 |
| 8 | 241.8 ± 102.3 | 68.4% | 31.6% | 0.008 | 0.222 | 0.652 |

TABLE 10
The Bliss independence score of the combination of
Cetuximab, SI-B001 with Osimertinib in the HCC827
xenograft mouse model (Example 6)

| Therapy 1 | Therapy 2 | Bliss score | P-value |
|---|---|---|---|
| Cetuximab | Osimertinib | 0.102 | <0.001 |
| SI-B001 9 mg/kg | Osimertinib | 0.115 | <0.001 |
| SI-B001 16 mg/kg | Osimertinib | 0.173 | <0.001 |
| SI-B001 28 mg/kg | Osimertinib | 0.250 | <0.001 |

TABLE 11
The Bliss independence score of the combination of
Cetuximab, SI-B001 with chemotherapy in the
HCC827 xenograft mouse model (Example 7)

| Therapy 1 | Therapy 2 | Bliss score | P-value |
|---|---|---|---|
| Cetuximab | Chemotherapy | 0.0002 | 0.053 |
| SI-B001 9 mg/kg | Chemotherapy | 0.026 | <0.001 |
| SI-B001 16 mg/kg | Chemotherapy | 0.052 | <0.001 |
| SI-B001 28 mg/kg | Chemotherapy | −0.008 | N.A. |

SEQUENCE LISTING
>Sequence ID NO 1: SI-B001, SI-1X6.4 bispecific
antibody heavy chain VH amino acid sequence with
CDRs.
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSS
>Sequence ID NO 2: SI-B001, SI-1x6.4 bispecific
heavy chain scFv domain amino acid sequence with
CDRs.
QVQLQESGGGLVKPGGSLRLSCAASGFTESSYWMSWVRQAPGKGLEWVAN
INRDGSASYYVDSVKGRFTISRDDAKNSLYLQMNSLRAEDTAVYYCARDR
GVGYFDLWGRGTLVTVSSGGGGSGGGGSGGGGSQSALTQPASVSGSPGQS
ITISCTGTSSDVGGYNFVSWYQQHPGKAPKLMIYDVSDRPSGVSDRFSGS
KSGNTASLIISGLQADDEADYYCSSYGSSSTHVIFGGGTKVTVL
>Sequence ID NO 3: SI-B001, SI-1X6.4 bispecific
antibody heavy chain full-length amino acid
sequence.
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGGG
GGSGGGGSQVQLQESGGGLVKPGGSLRLSCAASGFTFSSYWMSWVRQAPG
KGLEWVANINRDGSASYYVDSVKGRFTISRDDAKNSLYLQMNSLRAEDTA
VYYCARDRGVGYFDLWGRGTLVTVSSGGGGSGGGGSGGGGSQSALTQPAS
VSGSPGQSITISCTGTSSDVGGYNFVSWYQQHPGKAPKLMIYDVSDRPSG
VSDRESGSKSGNTASLIISGLQADDEADYYCSSYGSSSTHVIFGGGTKVT
VL
>Sequence ID NO 4: SI-B001, SI-1x6.4 bispecific
antibody light chain VL amino acid sequence with
CDRs.
DILLTQSPVILSVSPGERVSFSCRASQSIGTNIHWYQQRINGSPRLLIKY
ASESISGIPSRFSGSGSGTDFTLSINSVESEDIADYYCQQNNNWPTTFGA
GTKLELK
>Sequence ID NO 5:
SI-B001, SI-1x6.4 bispecific antibody light
chain full-length amino acid sequence.
DILLTQSPVILSVSPGERVSFSCRASQSIGTNIHWYQQRINGSPRLLIKY
ASESISGIPSRFSGSGSGTDFTLSINSVESEDIADYYCQQNNNWPTTFGA
GTKLELKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSENRGEC

What is claimed is:

1. A method for treating cancer in a subject comprising, administering to the subject a bispecific antibody having a binding specificity to EGFR and HER3 and a therapeutic agent, wherein the therapeutic agent comprises a tyrosine kinase inhibitor (TKI), an alkylating agent, an anti-metabolite, an anti-microtubule agent, a cytotoxic antibiotic, a topoisomerase inhibitor, a chemoprotectant, or a combination thereof.

2. The method of claim 1, wherein the bispecific antibody comprises 3 complementary determining regions (CDRs) of SEQ ID NO: 1, 3 CDRs of SEQ ID NO: 2, or 3 CDRs of SEQ ID NO: 4

3. The method of claim 1, wherein the bispecific antibody comprises a heavy chain variable region (VH) having an amino acid sequence with at least 98% sequence identity to SEQ ID NO: 1, a heavy chain scFv domain having an amino acid sequence with at least 98% sequence identity to SEQ ID NO: 2, and a light chain variable region (VL) having an amino acid sequence with at least 98% sequence identity to SEQ ID NO: 4

4. The method of claim 1, wherein the bispecific antibody comprises a heavy chain and a light chain, wherein the heavy chain comprises an amino acid sequence with at least 98% sequence identity to SEQ ID NO: 3 and the light chains comprises an amino acid sequence with at least 98% sequence identity to SEQ ID NO: 5.

5. The method of claim 1, wherein the therapeutic agent comprises osimertinib, paclitaxel, docetaxel, irinotecan, carboplatin, pemetrexed, cisplatin, or a combination thereof.

6. The method of claim 1, wherein the bispecific antibody and the therapeutic agent are administered simultaneously or sequentially as one treatment session.

7. The method of claim 1, wherein the bispecific antibody and the therapeutic agent is separately administered to the subject in alternating treatment session.

8. The method claim 1, wherein the bispecific antibody is administered in a first treatment session and the therapeutic agent is administered in a second treatment session, wherein the antibody is administered once a week (Q1W), every two weeks (Q2W), every three weeks (Q3W), or once a week for two weeks every three weeks (D1D8, Q3W), and wherein the antibody is administered at a fixed dose, a dose by mg/kg, or a dose by mg/m2.

9. The method of claim 8, wherein the duration of the first treatment session is from about 7 days to about 728 days.

10. The method of claim 8, wherein the duration of the second treatment session is from about 1 day to about 728 days.

11. The method of claim 8, wherein the gap between the first treatment session and the second treatment session is from about 7 to about 21 days.

12. The method of claim 1, wherein the bispecific antibody is administered at a dose of at least about 0.3 mg/kg, about 1.2 mg/kg, about 3.0 mg/kg, about 6.0 mg/kg, about 9.0 mg/kg, about 12.0 mg/kg, about 14.0 mg/kg, about 16.0 mg/kg, about 21.0 mg/kg or about 28.0 mg/kg.

13. The method of claim 1, wherein the therapeutic agent is administrated at a dose from about 6.0 mg/Kg to about 28.0 mg/Kg.

14. The method of claim 1, wherein the tyrosine kinase inhibitor (TKI) comprises Erlotinib, Gefitinib, Icotinib, AZD3759, Sapatinib, Afatinib, Dacomitinib, Deratinib, Poziotinib, Tarlox-TKI, Osimertinib, Nazartinib, Olmutinib, Rociletinib, Naquotinib, Lazertinib, EA1045, CLN081, AZ5104, Mobocertinib, its derivative or a combination thereof.

15. The method of claim 1, wherein the alkylating agent comprises Busulfan, Cyclophosphamide, Temozolomide, Carboplatin, Cisplatin, or a combination thereof.

16. The method of claim 1, wherein the anti-metabolite comprises 6-mercaptopurine, Fludarabine, 5-fluorouracil, Gemcitabine, Cytarabine, Pemetrexed, Methotrexate, its derivative or a combination thereof.

17. The method of claim 1, wherein the anti-microtubule agent comprises Docetaxel, Eribulin, Ixabepilone, Paclitaxel, Vinblastine, its derivative, or a combination thereof.

18. The method of claim 1, wherein the cytotoxic antibiotics comprises Dactinomycin, Bleomycin, Daunorubicin, Doxorubicin, its derivative, or a combination thereof.

19. The method of claim 1, wherein the topoisomerase inhibitor comprises Etoposide, Irinotecan, Topotecan, its derivative or a combination thereof.

20. The method of claim 1, wherein the chemoprotectant comprises Leucovorin or its derivative thereof.

21. The method of claim 1, wherein the therapeutic agent comprises Osimertinib, and wherein Osimertinib is administered at a dose of at least about 40 mg/kg, about 80 mg/kg, about 120 mg/kg, or about 160 mg/kg.

22. The method of claim 1, wherein the therapeutic agent comprises Carboplatin, and wherein Carboplatin is administered at a dose of at least about 200 mg/m2, about 250 mg/m2, about 300 mg/m2, about 360 mg/m2, about 400 mg/m2, about AUC 5 mg/ml/min, about AUC 6 mg/ml/min, or about 7 mg/ml/min.

23. The method of claim 1, wherein the therapeutic agent comprises Cisplatin, and wherein Cisplatin is administered at a dose of at least about 15 mg/m2, about 20 mg/m2, about 30 mg/m2, about 50 mg/m2, about 75 mg/m2, about 100 mg/m2, or about 120 mg/m2.

24. The method of claim 1, wherein the therapeutic agent comprises Pemetrexed, and wherein Pemetrexed is administered at a dose of at least about 250 mg/m2, about 500 mg/m2, or about 750 mg/m2.

25. The method of claim 1, wherein the therapeutic agent comprises Paclitaxel, and wherein Paclitaxel is administered at a dose of at least about 40 mg/m2, about 80 mg/m2, about 135 mg/m2, or about 175 mg/m2.

26. The method of claim 1, wherein the therapeutic agent comprises docetaxel, and wherein docetaxel is administered at a dose of at least about 35 mg/m2 D1D8Q3W.

27. The method of claim 1, wherein the bispecific antibody and the therapeutic agent are administered simultaneously and sequentially.

28. The method of claim 1, wherein the bispecific antibody is administered at a separate time from the therapeutic agent.

29. The method of claim 1, wherein the cancer comprises a solid tumor that tests positive for EGFR expression and is selected from the group consisting of: lung adenocarcinoma, head/neck squamous cell cancer, rectal cancer, colon cancer, squamous cell lung cancer, thyroid cancer, bladder cancer, melanoma, cervical cancer, prostate cancer, breast cancer, uterine/endometrial cancer, pancreatic cancer, ovarian cancer, and papillary kidney cancer.

30. A therapeutic composition comprising, a combination of a bispecific antibody having a binding specificity to EGFR and HER3 and a therapeutic agent, wherein the therapeutic agent comprises a tyrosine kinase inhibitor (TKI), an alkylating agent, an anti-metabolite, an anti-microtubule agent, a cytotoxic antibiotic, a topoisomerase inhibitor, a chemoprotectant, or a combination thereof.

31. The therapeutic composition of claim 30, wherein the bispecific antibody comprises 3 complementary determining regions (CDRs) of SEQ ID NO: 1, 3 CDRs of SEQ ID NO: 2, or 3 CDRs of SEQ ID NO: 4, and wherein the therapeutic agent comprises Osimertinib, Carboplatin, Cisplatin, Pemetrexed, Paclitaxel, its derivative, or a combination thereof.

32. The therapeutic composition of claim 30, wherein the bispecific antibody and the therapeutic agent are in the form of a pharmaceutical formulation to be administered simultaneously, sequentially, or concurrently.

33. A kit comprising a first container, a second container and a package insert,
wherein the first container comprises at least one dose of a first therapeutic composition comprising a bispecific antibody having a binding specificity to EGFR and HER3, the second container comprises at least one dose of a second therapeutic composition comprising a therapeutic agent, and the package insert comprises instructions for treating a subject for cancer using the first and the second therapeutic compositions, and wherein the therapeutic agent comprises a tyrosine kinase inhibitor (TKI), an alkylating agent, an anti-metabolite, an anti-microtubule agent, a cytotoxic antibiotic, a topoisomerase inhibitor, a chemoprotectant, or a combination thereof.

34. The kit of claim 33, wherein the instructions state that the first and the second therapeutic compositions are intended for use in treating a subject having a cancer that tests positive for EGFR expression.


SEQUENCE LISTING (5 sequences)

SEQ ID NO: 1 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSS

SEQ ID NO: 2 - protein, 244 aa, synthetic construct
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 3 - protein, 702 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 4 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 5 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC